(19) United States
(12) Patent Application Publication
(10) Pub. No.: US 2019/0309326 A1
(43) Pub. Date: Oct. 10, 2019

(54) DUAL OVERLAPPING ADENO-ASSOCIATED VIRAL VECTOR SYSTEM FOR EXPRESSING ABCA4

(71) Applicant: Oxford University Innovation Limited, Oxford (GB)

(72) Inventors: Robert MACLAREN, Oxford (GB); Michelle MCCLEMENTS, Oxford (GB)

(21) Appl. No.: 16/309,428

(22) PCT Filed: Jun. 14, 2017
(86) PCT No.: PCT/GB2017/051741

§ 371 Date: Dec. 12, 2018

(30) Foreign Application Priority Data
Jun. 15, 2016 (GB) 1610448.1
May 5, 2017 (GB) 1707261.2

(51) Int. Cl. C12N 15/86; A61K 35/761; A61K 9/00; A61P 27/02

(52) U.S. Cl. CPC C12N 15/86; A61K 35/761; A61K 48/00; A61P 27/02; C12N 2750/14143; A61K 9/0048

(57) ABSTRACT

The present invention provides an adeno-associated viral (AAV) vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1; wherein the second nucleic acid sequence comprises a sequence of contiguous nucleotides corresponding to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1. Also provided are uses of AAV vector systems in the prevention or treatment of disease.

### FIELD OF THE INVENTION

The present invention relates to adeno-associated viral (AAV) vector systems and AAV vectors for expressing human ABCA4 protein in a target cell. The AAV vector systems and AAV vectors of the invention may be used in preventing or treating diseases associated with degradation of retinal cells such as Stargardt disease.

### BACKGROUND TO THE INVENTION

Stargardt disease is an inherited disease of the retina that can lead to blindness through the destruction of light-sensing photoreceptor cells in the eye. The disease commonly presents in childhood leading to blindness in young people.

The most common form of Stargardt disease is a recessive disorder linked to mutations in the gene encoding the protein ATP Binding Cassette, sub-family A, member 4 (ABCA4). ABCA4 is a large, transmembrane protein that plays a role in the recycling of light-sensitive pigments in retinal cells. In Stargardt disease, mutations in the ABCA4 gene lead to a lack of functional ABCA4 protein in retinal cells. This in turn leads to the formation and accumulation of bisretinoid by-products, producing toxic granules of lipofuscin in Retinal Pigment Epithelial (RPE) cells. This causes degradation and eventual destruction of the RPE cells, which leads to loss of photoreceptor cells causing progressive loss of vision and eventual blindness.

Gene therapy holds promise as a treatment for Stargardt disease. The aim is to correct the deficiency underlying the disease by using a vector to introduce a functional ABCA4 gene into the affected photoreceptor cells, thus restoring ABCA4 function.

Vectors derived from adeno-associated virus (AAV) are currently under investigation for retinal gene therapy. AAV is a small virus that presents very low immunogenicity and is not associated with any known human disease. The lack of an associated inflammatory response means that AAV does not cause retinal damage when injected into the eye.

However, the size of the AAV capsid imposes a limit on the amount of DNA that can be packaged within it. The AAV genome is approximately 4.7 kilobases (kb) in size, and it is believed that the corresponding upper size limit for DNA packaging in AAV is approximately 5 kb (Wu et al., Molecular Therapy, vol. 18, No. 1, January 2010). The coding sequence of the ABCA4 gene is approximately 6.8 kb in size (with further genetic elements being required for gene expression), making it too large to be incorporated into a standard AAV vector.

A number of approaches to overcome this upper size limit and express large genes such as ABCA4 from AAV vectors have been trialled. These approaches include “oversize” vector approaches and “dual” vector approaches.

### “Oversize” Vectors

A number of attempts have been made to force genes considerably larger than the native 4.7 kb genome into AAV vectors, with some success in transducing target cells. By way of example, Allocca et al. (J. Clin. Invest. vol. 118, No. 5, May 2008) prepared oversize AAV vectors packaging the murine ABCA4 and human MYO7A genes and demonstrated protein expression following transduction of mouse retinal cells. However, while it was proposed by Allocca et al. that certain AAV capsids could accommodate up to 8.9 kb, subsequent studies have found that the “oversize” approach does not in fact overcome the packaging upper size limit, but rather leads to truncation of the transgene in a random manner, providing a heterogeneous population of AAV vectors each comprising a fragment of the transgene (Dong et al., Molecular Therapy, vol. 18, No. 1, January 2010). It is believed that a proportion of oversize vectors in a given population package large enough fragments of the oversized transgene such that regions of overlap between the fragments exist, allowing re-assembly into a full length gene following transduction of a target cell. However, this method is unpredictable and inefficient, with the lack of packaging control and subsequent failure of recombination providing a significant barrier to consistent, detectable success.

### “Dual” Vectors

An alternative approach has been to prepare dual vector systems, in which a transgene larger than the approximately 5 kb limit is split approximately in half into two separate vectors of defined sequence: an “upstream” vector containing the 5′ portion of the transgene, and a “downstream” vector containing the 3′ portion of the transgene. Transduction of a target cell by both upstream and downstream vectors allows a full-length transgene to be re-assembled from the two fragments using a variety of intracellular mechanisms.

In a so-called “trans-splicing” dual vector approach, a splice-donor signal is placed at the 3′ end of the upstream transgene fragment and a splice-acceptor signal placed at the 5′ end of the downstream transgene fragment. Upon transduction of a target cell by the dual vectors, inverted terminal repeat (ITR) sequences present in the AAV genome mediate head-to-tail concatermerisation of the transgene fragments and trans-splicing of the transcripts results in the production of a full-length mRNA sequence, allowing full-length protein expression.

An alternative dual vector system uses an “overlapping” approach. In an overlapping dual vector system, part of the coding sequence at the 3′ end of the upstream coding sequence portion overlaps with a homologous sequence at the 5′ of the downstream coding sequence portion. Upon transduction of a target cell by upstream and downstream vectors, homologous recombination between the upstream and downstream portions of coding sequence allows for the recreation of a full-length transgene, from which a corresponding mRNA can be transcribed and full-length protein expressed.

WO 2014/170480 describes the generation of a dual AAV vector system encoding human ABCA4 protein.

There is therefore a need in the art for alternative and/or improved AAV vector systems encoding the ABCA4 protein and suitable for use in gene therapy.

### SUMMARY OF INVENTION

The present invention addresses the above prior art problems by providing adeno-associated viral (AAV) vector systems as described in the claims.

Advantageously, the AAV vector system of the invention provides surprisingly high levels of expression of full-length ABCA4 protein in transduced cells, with limited production of unwanted truncated fragments of ABCA4.

In one aspect, the invention provides an AAV vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1; wherein the second nucleic acid sequence comprises a sequence of contiguous nucleotides corresponding to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1.

The region of sequence overlap may be between 20 and 550 nucleotides in length; preferably between 50 and 250 nucleotides in length; more preferably between 175 and 225 nucleotides in length; and most preferably between 195 and 215 nucleotides in length.

The region of sequence overlap may also comprise at least about 50 contiguous nucleotides of a nucleic acid sequence corresponding to nucleotides 3598 to 3805 of SEQ ID NO: 1; preferably at least about 75 contiguous nucleotides; more preferably at least about 100 contiguous nucleotides; even more preferably at least about 150 contiguous nucleotides; and most preferably at least about 200 contiguous nucleotides.

In one embodiment, the first nucleic acid sequence comprises a sequence of contiguous nucleotides consisting of nucleotides 105 to 3597 of SEQ ID NO: 1. In one embodiment, the second nucleic acid sequence comprises a sequence of contiguous nucleotides consisting of nucleotides 3806 to 6926 of SEQ ID NO: 1.

In one embodiment, the first nucleic acid sequence comprises a sequence of contiguous nucleotides consisting of nucleotides 105 to 3597 of SEQ ID NO: 2. In one embodiment, the second nucleic acid sequence comprises a sequence of contiguous nucleotides consisting of nucleotides 3806 to 6926 of SEQ ID NO: 2.

In one embodiment, the region of sequence overlap comprises at least about 20 contiguous nucleotides of a nucleic acid sequence consisting of nucleotides 3598 to 3805 of SEQ ID NO: 1. In one embodiment, the region of sequence overlap comprises at least about 20 contiguous nucleotides of a nucleic acid sequence consisting of nucleotides 3598 to 3805 of SEQ ID NO: 2.

In one embodiment, the region of sequence overlap comprises at least about 50 contiguous nucleotides of a nucleic acid sequence consisting of nucleotides 3598 to 3805 of SEQ ID NO: 1; preferably at least about 75 contiguous nucleotides; more preferably at least about 100) contiguous nucleotides; even more preferably at least about 150 contiguous nucleotides; and most preferably at least about 200 contiguous nucleotides. In one embodiment, the region of sequence overlap comprises at least about 50 contiguous nucleotides of a nucleic acid sequence consisting of nucleotides 3598 to 3805 of SEQ ID NO: 2; preferably at least about 75 contiguous nucleotides; more preferably at least about 100 contiguous nucleotides; even more preferably at least about 150 contiguous nucleotides; and most preferably at least about 200 contiguous nucleotides.

In one embodiment, the first nucleic acid sequence comprises a sequence of contiguous nucleotides corresponding to nucleotides 105 to 3805 of SEQ ID NO: 1; and the second nucleic acid sequence comprises a sequence of contiguous nucleotides corresponding to nucleotides 3598 to 6926 of SEQ ID NO: 1.

In one embodiment, the first nucleic acid sequence comprises a sequence of contiguous nucleotides consisting of nucleotides 105 to 3805 of SEQ ID NO: 1; and the second nucleic acid sequence comprises a sequence of contiguous nucleotides consisting of nucleotides 3598 to 6926 of SEQ ID NO: 1.

In one embodiment, the first nucleic acid sequence comprises a sequence of contiguous nucleotides consisting of nucleotides 105 to 3805 of SEQ ID NO: 2; and the second nucleic acid sequence comprises a sequence of contiguous nucleotides consisting of nucleotides 3598 to 6926 of SEQ ID NO: 2.

The first AAV vector may comprise a GRK1 promoter operably linked to the 5′ end portion of an ABCA4 coding sequence (CDS).

The first nucleic acid sequence may comprise an untranslated region (UTR) located upstream of the 5′ end portion of an ABCA4 coding sequence (CDS).

The second nucleic acid sequence may comprise a post-transcriptional response element (PRE); preferably a Woodchuck hepatitis virus post-transcriptional response element (WPRE).

The second nucleic acid sequence may comprise a bovine Growth Hormone (bGH) poly-adenylation sequence.

In another aspect, the invention provides a method for expressing a human ABCA4 protein in a target cell, the method comprising the steps of: transducing the target cell with the first AAV vector and the second AAV vector as defined above, such that a functional ABCA4 protein is expressed in the target cell.

In a further aspect, the invention provides an AAV vector comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1. In one embodiment, this AAV vector comprises the nucleic acid sequence of SEQ ID NO: 9. In one embodiment, the 5′ end portion of an ABCA4 CDS consists of nucleotides 105 to 3805 of SEQ ID NO: 1. In one embodiment, the 5′ end portion of an ABCA4 CDS consists of nucleotides 105 to 3805 of SEQ ID NO: 2.

In a further aspect, the invention provides an AAV vector comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1. In one embodiment, this AAV vector comprises the nucleic acid sequence of SEQ ID NO: 10. In one embodiment, the 3′ end portion of an ABCA4 CDS consists of nucleotides 3598 to 6926 of SEQ ID NO: 1. In one embodiment, the 3′ end portion of an ABCA4 CDS consists of nucleotides 3598 to 6926 of SEQ ID NO: 2.

In another aspect, the invention provides a nucleic acid comprising the first nucleic acid sequence as defined above.

In another aspect, the invention provides a nucleic acid comprising the second nucleic acid sequence as defined above.

Also provided by the invention are a nucleic acid comprising the nucleic acid sequence of SEQ ID NO: 9, and a nucleic acid comprising the nucleic acid sequence of SEQ ID NO: 10.

In a further aspect, the invention provides a kit comprising the AAV vector system as described above, or the upstream AAV vector and the downstream AAV vector as described above.

The invention also provides a kit comprising a nucleic acid comprising the first nucleic acid sequence and a nucleic acid comprising the second nucleic acid sequence, as described above, or a nucleic acid comprising the nucleic acid sequence of SEQ ID NO: 9 and a nucleic acid comprising the nucleic acid sequence of SEQ ID NO: 10, as described above.

In yet a further aspect, the invention provides a pharmaceutical composition comprising the AAV vector system as described above and a pharmaceutically acceptable excipient.

In a yet a further aspect, the invention provides an AAV vector system as described above, a kit as described above, or a pharmaceutical composition as described above, for use in preventing or treating disease characterised by degradation of retinal cells; preferably for use in preventing or treating Stargardt disease.

In another aspect, the invention provides a method for preventing or treating a disease characterised by degradation of retinal cells, such as Stargardt disease, comprising administering to a subject in need thereof an effective amount of an AAV vector system as described above, a kit as described above, or a pharmaceutical composition as described above.

In another aspect, the invention provides an AAV vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3597 of SEQ ID NO: 1; wherein the second nucleic acid sequence comprises a sequence having at least 90% (e.g. at least 90, 95, 96, 97, 98, 99, 99.1, 99.2, 99.3, 99.4, 99.5, 99.6, 99.7, 99.8, 99.9 or 100%/o) sequence identity to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 3805 of SEQ ID NO: 1.

In another aspect, the invention provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3805 of SEQ ID NO: 1, and %% herein the 3′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 6926 of SEQ ID NO: 1.

In another aspect, the invention provides an AAV vector comprising a nucleic acid sequence comprising a 5′ end portion of an ABCA4 CDS, wherein the 5′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100° %) sequence identity to nucleotides 105 to 3805 of SEQ ID NO: 1.

In another aspect, the invention provides an AAV vector comprising a nucleic acid sequence comprising a 3′ end portion of an ABCA4 CDS, wherein the 3′ end portion of an ABCA4 CDS consists of a sequence having at least 90%/o (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 6926 of SEQ ID NO: 1.

In another aspect, the invention provides an AAV vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3597 of SEQ ID NO: 2; wherein the second nucleic acid sequence comprises a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3806 to 6926 of SEQ ID NO: 2; wherein the first nucleic acid sequence and the second nucleic acid sequence each comprise a region of sequence overlap with the other; and wherein the region of sequence overlap comprises at least about 20 contiguous nucleotides of a nucleic acid sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 3805 of SEQ ID NO: 2.

In another aspect, the invention provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence having at least 90%6 (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3805 of SEQ ID NO: 2, and wherein the 3′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 6926 of SEQ ID NO: 2.

In another aspect, the invention provides an AAV vector comprising a nucleic acid sequence comprising a 5′ end portion of an ABCA4 CDS, wherein the 5′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 105 to 3805 of SEQ ID NO: 2.

In another aspect, the invention provides an AAV vector comprising a nucleic acid sequence comprising a 3′ end portion of an ABCA4 CDS, wherein the 3′ end portion of an ABCA4 CDS consists of a sequence having at least 90% (e.g. at least 90, 95, 96, 97, 98, 99, 99.1, 99.2, 99.3, 99.4, 99.5, 99.6, 99.7, 99.8, 99.9 or 100%) sequence identity to nucleotides 3598 to 6926 of SEQ ID NO: 2.

Also provided by the invention are a nucleic acid comprising a nucleic acid sequence having at least 90% (e.g. at least 90, 95, 96, 97, 98, 99, 99.1, 99.2, 99.3, 99.4, 99.5, 99.6, 99.7, 99.8 or 99.9%) sequence identity to SEQ ID NO: 9, and a nucleic acid comprising a nucleic acid sequence having at least 90% (e.g. at least 90, 95, 96, 97, 98, 99, 99.1, 99.2, 99.3, 99.4, 99.5, 99.6, 99.7, 99.8 or 99.9%) sequence identity to SEQ ID NO: 10.

### DESCRIPTION OF FIGURES

FIG. 1. Upstream and downstream transgene structures that combine to form a complete ABCA4 transgene.

FIG. 2. ABCA4 protein detection in Abca4^{-/-} retinae 6 weeks post-injection with dual vector variant C with (5′C) and without (C) the extra UTR sequence. Units represent fold increase relative to uninjected KO samples. Error bars represent SEM. One-way ANOVA. Tukey post-hoc. p=**0.009.

FIG. 3. Representation of the ABCA4 CDS contained in upstream and downstream transgenes that make up overlap variants A, B, C, D, E, F and X. (a) ABCA4 protein detection following transduction with the different overlap zone vector variants in vitro and (b) in vivo. Units represent fold increase relative to untreated samples (−=untreated HEK293T cells; KO=uninjected Abca4^{-/-} retinae). Error bars represent SEM. One-way ANOVA, Tukey post-hoc analyses revealed that in vitro, dual vector variants B and C generated significantly more ABCA4 protein than all other samples but there was no significant difference between B and C. In vivo, dual vector variant C generated significantly more ABCA4 protein than all other variants (except B).

FIG. 4. (a) Truncated ABCA4 protein variants detectable in HEK293T cells treated with unrecombined downstream vectors; (b) truncated and full length ABCA4 protein detected in Abca4^{-/-} retinae samples injected with dual vector 5′B or 5′C; (c) Table presents percentage full length ABCA4 present in the total ABCA4 protein population detected by western blot of injected retinae(d) difference in fold change of ABCA4 expression between overlap C dual vector variant injected retinae and overlap B dual vector variant injected retinae at transcript and protein level. Error bars represent SEM.

FIG. 5. a) Overlap C sequence with out-of-frame AUG codons prior to an in-frame AUG codon; b) predicted secondary structures of overlap zones C and B.

FIG. 6. Staining of ABCA4 (green) in the outer segments of photoreceptor cells in an Abca4^{-/-}, retina harvested 6 weeks post-injection. HCN1 (red) staining marks the inner segments. Staining example of native Abca4 localisation in a WT retina is also included plus evidence of absence of staining in an uninjected Abca4^{-/-} retina.

FIG. 7. Abca4/ABCA4 (green) and Hcn1 (red) staining in wild-type (WT) and Abca4^{-/-} eyes.

FIG. 8. Abca4/ABCA4 (green) and rhodopsin (red) staining in photoreceptor cell outer segments in wild-type (WT) and Abca4^{-/-} eyes.

FIG. 9. Abca4/ABCA4 (green) and rhodopsin (red) apical RPE staining in wild-type (WT) and Abca4^{-/-} eyes.

FIG. 10. Diagram of example overlapping vectors.

FIG. 11. The normal retinoid cycle is shown on the left-hand side of the diagram. The generation of bisretinoids and A2E that occurs to an enhanced degree in Abca4 deficient mice and humans is shown on the right. The molecules highlighted in boxes on the right-hand side of the diagram were assessed in Abca4^{-/-}, mice. (Example 6.)

FIG. 12. Levels of bisretinoids and A2E isoforms in paired eyes for 13 Abca4^{-/-} mice that received either sham or treatment injection. A significant decrease in bisretinoid and A2E levels was observed between sham and treatment eyes (p=0.017, F=5.849). Furthermore, for all bisretinoid and A2E measurements, the lowest levels were seen in the dual vector treated eyes. (Example 6.)

### LIST OF SEQUENCES

SEQ ID NO: 1 Human ABCA4 nucleic acid sequence. SEQ ID NO: 1 is identical to NCBI Reference Sequence NM_000350.2.
SEQ ID NO: 2 Human ABCA4 nucleic acid sequence variant. SEQ ID NO: 2 is identical to SEQ ID NO: 1 with the exception of the following mutations: nucleotide 1640 G>T, nucleotide 5279 G>A, nucleotide 6173 T>C.
SEQ ID NO: 3 Example upstream vector sequence, comprising ITR, promoter, CDS, ITR.
SEQ ID NO: 4 Example downstream vector sequence, comprising ITR. CDS, post-transcriptional response element, poly-adenylation sequence, ITR.
SEQ ID NO: 5 GRK1 promoter sequence.
SEQ ID NO: 6 UTR sequence.
SEQ ID NO: 7 Woodchuck Hepatitis Virus post-transcriptional response element.
SEQ ID NO: 8 Bovine Growth Hormone poly-adenylation sequence.
SEQ ID NO: 9 Example partial upstream vector sequence, comprising promoter, CDS.
SEQ ID NO: 10 Example partial downstream vector sequence, comprising CDS, post-transcriptional response element, poly-adenylation sequence.

### DETAILED DESCRIPTION

Viral vectors are derived from wildtype viruses which are modified using recombinant nucleic acid technologies to incorporate a non-native nucleic acid sequence (or transgene) into the viral genome. The ability of viruses to target and infect specific cells is used to deliver the transgene into a target cell, leading to the expression of the gene and the production of the encoded gene product.

The present invention relates to vectors derived from adeno-associated virus (AAV).

In a first aspect, the invention provides an adeno-associated viral (AAV) vector system for expressing a human ABCA4 protein in a target cell, the AAV vector system comprising a first AAV vector comprising a first nucleic acid sequence and a second AAV vector comprising a second nucleic acid sequence; wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the first nucleic acid sequence comprises a sequence of contiguous nucleotides corresponding to nucleotides 105 to 3597 of SEQ ID NO: 1; wherein the second nucleic acid sequence comprises a sequence of contiguous nucleotides corresponding to nucleotides 3806 to 6926 of SEQ ID NO: 1; wherein the first nucleic acid sequence and the second nucleic acid sequence each comprise a region of sequence overlap with the other; and wherein the region of sequence overlap comprises at least about 20 contiguous nucleotides of a nucleic acid sequence corresponding to nucleotides 3598 to 3805 of SEQ ID NO: 1.

AAV vectors in general are well known in the art and a skilled person will be familiar with general techniques suitable for their preparation from his common general knowledge in the field. The skilled person's knowledge will include techniques suitable for incorporating a nucleic acid sequence of interest into the genome of an AAV vector.

The term “AAV vector system” is used to embrace the fact that the first and second AAV vectors are intended to work together in a complementary fashion.

The first and second AAV vectors of the AAV vector system of the invention together encode an entire ABCA4 transgene. Thus, expression of the encoded ABCA4 transgene in a target cell requires transduction of the target cell with both first (upstream) and second (downstream) vectors.

The AAV vectors of the AAV vector system of the invention are typically in the form of AAV particles (also referred to as virions). An AAV particle comprises a protein coat (the capsid) surrounding a core of nucleic acid, which is the AAV genome. The present invention also encompasses nucleic acid sequences encoding AAV vector genomes of the AAV vector system described herein.

SEQ ID NO: 1 is the human ABCA4 nucleic acid sequence corresponding to NCBI Reference Sequence NM_000350.2. SEQ ID NO: 1 is identical to NCBI Reference Sequence NM_000350.2. The ABCA4 coding sequence spans nucleotides 105 to 6926 of SEQ ID NO: 1.

The first AAV vector comprises a first nucleic acid sequence comprising a 5′ end portion of an ABCA4 CDS. A 5′ end portion of an ABCA4 CDS is a portion of the ABCA4 CDS that includes its 5′ end. Because it is only a portion of a CDS, the 5′ end portion of an ABCA4 CDS is not a full-length (i.e. is not an entire) ABCA4 CDS. Thus, the first nucleic acid sequence (and thus the first AAV vector) does not comprise a full-length ABCA4 CDS.

The second AAV vector comprises a second nucleic acid sequence comprising a 3′ end portion of an ABCA4 CDS. A 3′ end portion of an ABCA4 CDS is a portion of the ABCA4 CDS that includes its 3′ end. Because it is only a portion of a CDS, the 3′ end portion of an ABCA4 CDS is not a full-length (i.e. is not an entire) ABCA4 CDS. Thus, the second nucleic acid sequence (and thus the second AAV vector) does not comprise a full-length ABCA4 CDS.

The 5′ end portion and 3′ end portion together encompass the entire ABCA4 CDS (with a region of sequence overlap, as discussed below). Thus, a full-length ABCA4 CDS is contained in the AAV vector system of the invention, split across the first and second AAV vectors, and can be reassembled in a target cell following transduction of the target cell with the first and second AAV vectors.

The first nucleic acid sequence as described above comprises a sequence of contiguous nucleotides corresponding to nucleotides 105 to 3597 of SEQ ID NO: 1. The ABCA4 CDS begins at nucleotide 105 of SEQ ID NO: 1.

The second nucleic acid sequence as described above comprises a sequence of contiguous nucleotides corresponding to nucleotides 3806 to 6926 of SEQ ID NO: 1.

In order to encompass the entire ABCA4 CDS, the first and second nucleic acid sequences each further comprise at least a portion of the ABCA4 CDS corresponding to nucleotides 3598 to 3805 of SEQ ID NO: 1, such that when the first and second nucleic acid sequences are aligned the entirety of ABCA4 CDS corresponding to nucleotides 3598 to 3805 of SEQ ID NO: 1 is encompassed. Thus, when aligned, the first and second nucleic acid sequences together encompass the entire ABCA4 CDS.

Furthermore, the first and second nucleic acid sequences comprise a region of sequence overlap allowing reconstruction of the entire ABCA4 CDS as part of a full-length transgene inside a target cell transduced with the first and second AAV vectors of the invention.

When the first and second nucleic acid sequences are aligned with each other, a region at the 3′ end of the first nucleic acid sequence overlaps with a corresponding region at the 5′ end of the second nucleic acid sequence. Thus, both the first and second nucleic acid sequences comprise a portion of the ABCA4 CDS that forms the region of sequence overlap.

The present inventors have found that particularly advantageous results are obtained when the region of overlap between the first and second nucleic acid sequences comprises at least about 20 contiguous nucleotides of the portion of the ABCA4 CDS corresponding to nucleotides 3598 to 3805 of SEQ ID NO: 1.

The region of overlap may extend upstream and/or downstream of said 20 contiguous nucleotides. Thus, the region of overlap may be more than 20 nucleotides in length.

The region of overlap may comprise nucleotides upstream of the position corresponding to nucleotide 3598 of SEQ ID NO: 1. Alternatively, or in addition, the region of overlap may comprise nucleotides downstream of the position corresponding to nucleotide 3805 of SEQ ID NO: 1.

Alternatively, the region of nucleic acid sequence overlap may be contained within the portion of the ABCA4 CDS corresponding to nucleotides 3598 to 3805 of SEQ ID NO: 1.

Thus, in one embodiment, the region of nucleic acid sequence overlap is between 20 and 550 nucleotides in length; preferably between 50 and 250 nucleotides in length; preferably between 175 and 225 nucleotides in length; preferably between 195 and 215 nucleotides in length.

In one embodiment, the region of nucleic acid sequence overlap comprises at least about 50 contiguous nucleotides of a nucleic acid sequence corresponding to nucleotides 3598 to 3805 of SEQ ID NO: 1; preferably at least about 75 contiguous nucleotides; preferably at least about 100 contiguous nucleotides; preferably at least about 150 contiguous nucleotides; preferably at least about 200 contiguous nucleotides; preferably all 208 contiguous nucleotides.

In a preferred embodiment, the region of nucleic acid sequence overlap commences at the nucleotide corresponding to nucleotide 3598 of SEQ ID NO: 1. The term “commences” means that the region of nucleic acid sequence overlap runs in the direction 5′ to 3′ starting from the nucleotide corresponding to nucleotide 3598 of SEQ ID NO: 1. Thus, in a preferred embodiment, the most 5′ nucleotide of the region of nucleic acid sequence overlap corresponds to nucleotide 3598 of SEQ ID NO: I.

In a further preferred embodiment, the region of nucleic acid sequence overlap between the first nucleic acid sequence and the second nucleic acid sequence vector corresponds to nucleotides 3598 to 3805 of SEQ ID NO: 1.

A further advantage of the present invention is that construction of dual AAV vectors comprising a region of nucleic acid sequence overlap as described above can advantageously reduce the level of translation of unwanted truncated ABCA4 peptides.

The problem of translation of truncated ABCA4 peptides may arise in dual AAV vector systems when translation is initiated from mRNA transcripts derived from the downstream vector only. In this regard, AAV ITRs such as the AAV2 5′ ITR may have promoter activity; this together with the presence in a downstream vector of WPRE and bGH poly-adenylation sequences (as discussed below) may lead to the generation of stable mRNA transcripts from unrecombined downstream vectors. The wild-type ABCA4 CDS carries multiple in-frame AUG codons in its downstream portion that cannot be substituted for other codons without altering the amino acid sequence. This creates the possibility of translation occurring from the stable transcripts, leading to the presence of truncated ABCA4 peptides.

In preferred embodiments of the invention wherein the region of nucleic acid sequence overlap commences at the nucleotide corresponding to nucleotide 3598 of SEQ ID NO: 1, the starting sequence of the overlap zone includes an out-of-frame AUG (start) codon in good context (regarding the potential Kozak consensus sequence) prior to an in-frame AUG codon in weaker context in order to encourage the translational machinery to initiate translation of unrecombined downstream-only transcripts from an out-of-frame site. In particularly preferred embodiments of the invention, there are in total four out-of-frame AUG codons in various contexts prior to the in-frame AUG. All of these will translate to a STOP codon within 10 amino acids, thus preventing the translation of unwanted truncated ABCA4 peptides.

Preferably, the first nucleic acid sequence comprises a sequence of contiguous nucleotides corresponding to nucleotides 105 to 3805 of SEQ ID NO: 1, and the second nucleic acid sequence comprises a sequence of contiguous nucleotides corresponding to nucleotides 3598 to 6926 of SEQ ID NO: 1, so encompassing the particularly preferred region of nucleic acid sequence overlap as described above.

Thus, in a preferred embodiment, the 5′ end portion of an ABCA4 CDS consists of a sequence of contiguous nucleotides corresponding to nucleotides 105 to 3805 of SEQ ID NO: 1, and the 3′ end portion of an ABCA4 CDS consists of a sequence of contiguous nucleotides corresponding to nucleotides 3598 to 6926 of SEQ ID NO: 1.

In a further preferred embodiment, the 5′ end portion of an ABCA4 CDS consists of nucleotides 105 to 3805 of SEQ ID NO: 1, and the 3′ end portion of an ABCA4 CDS consists of nucleotides 3598 to 6926 of SEQ ID NO: 1.

Thus, in a preferred embodiment, the invention provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of a sequence of contiguous nucleotides corresponding to nucleotides 105 to 3805 of SEQ ID NO: 1, and wherein the 3′ end portion of an ABCA4 CDS consists of a sequence of contiguous nucleotides corresponding to nucleotides 3598 to 6926 of SEQ ID NO: 1.

In a further preferred embodiment, the invention provides an AAV vector system for expressing a human ABCA4 protein in a target cell, the AAV vector system comprising a first AAV vector comprising a first nucleic acid sequence and a second AAV vector comprising a second nucleic acid sequence, wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS; wherein the 5′ end portion of an ABCA4 CDS consists of nucleotides 105 to 3805 of SEQ ID NO: 1, and wherein the 3′ end portion of an ABCA4 CDS consists of nucleotides 3598 to 6926 of SEQ ID NO: 1.

In accordance with the term “consists of”, in embodiments wherein the 5′ end portion of an ABCA4 CDS and the 3′ end portion of an ABCA4 CDS consist of specific sequences of contiguous nucleotides as described above, then the first nucleic acid sequence and the second nucleic acid sequence each do not comprise any additional ABCA4 CDS.

Typically, each of the first AAV vector and the second AAV vector comprises 5′ and 3′ Inverted Terminal Repeats (ITRs).

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. AAV ITRs are believed to aid concatemer formation in the nucleus of an AAV-infected cell, for example following the conversion of single-stranded vector DNA into double-stranded DNA by the action of host cell DNA polymerases. The formation of such episomal concatemers may serve to protect the vector construct during the life of the host cell, thereby allowing for prolonged expression of the transgene in vivo.

Thus, in one embodiment, the ITRs are AAV ITRs (i.e. ITR sequences derived from ITR sequences found in an AAV genome).

The first and second AAV vectors of the AAV vector system of the invention together comprise all of the components necessary for a fully functional ABCA4 transgene to be re-assembled in a target cell following transduction by both vectors. A skilled person will be aware of additional genetic elements commonly used to ensure transgene expression in a viral vector-transduced cell. These may be referred to as expression control sequences. Thus, the AAV vectors of the AAV viral vector system of the invention typically comprise expression control sequences (e.g. comprising a promoter sequence) operably linked to the nucleotide sequences encoding the ABCA4 transgene.

5′ expression control sequences components are suitably located in the first (“upstream”) AAV vector of the viral vector system, while 3′ expression control sequences are suitably located in the second (“downstream”) AAV vector of the viral vector system.

Thus, the first AAV vector typically comprises a promoter operably linked to the 5′ end portion of an ABCA4 CDS. The promoter is required by its nature to be located 5′ to the ABCA4 CDS, hence its location in the first AAV vector.

Any suitable promoter may be used, the selection of which may be readily made by the skilled person. The promoter sequence may be constitutively active (i.e. operational in any host cell background), or alternatively may be active only in a specific host cell environment, thus allowing for targeted expression of the transgene in a particular cell type (e.g. a tissue-specific promoter). The promoter may show inducible expression in response to presence of another factor, for example a factor present in a host cell. In any event, where the vector is administered for therapy, it is preferred that the promoter should be functional in the target cell background.

In some embodiments, it is preferred that the promoter shows retinal-cell specific expression in order to allow for the transgene to only be expressed in retinal cell populations. Thus, expression from the promoter may be retinal-cell specific, for example confined only to cells of the neurosensory retina and retinal pigment epithelium.

An example promoter suitable for use in the present invention is the chicken beta-actin (CBA) promoter, optionally in combination with a cytomegalovirus (CMV) enhancer element. Another example promoter for use in the invention is a hybrid CBA/CAG promoter, for example the promoter used in the rAVE expression cassette (GeneDetect.com).

Examples of promoters based on human sequences that would induce retina-specific gene expression include rhodopsin kinase for rods and cones, PR2.1 for cones only, and RPE65 for the retinal pigment epithelium.

The present inventors have found that particularly advantageous levels of gene expression may be achieved using a GRK1 promoter. Thus, in a preferred embodiment, the promoter is a human rhodopsin kinase (GRK1) promoter.

The GRK1 promoter sequence of the invention may be 199 nucleotides in length and comprise nucleotides −112 to +87 of the GRK1 gene. In a preferred embodiment, the promoter comprises the nucleic acid sequence of SEQ ID NO: 5 or a variant thereof having at least 90% (e.g. at least 90, 95, 96, 97, 98, 99, 99.1, 99.2, 99.3, 99.4 or 99.5, 99.6, 99.7, 99.8 or 99.9%) sequence identity.

The first AAV vector may comprise an untranslated region (UTR) located between the promoter and the upstream ABCA4 nucleic acid sequence (i.e. a 5′ UTR).

Any suitable UTR sequence may be used, the selection of which may be readily made by the skilled person.

The UTR may comprise one or more of the following elements: a Gallus gallus β-actin (CBA) intron 1 fragment, an Oryctolagus cuniculus β-globin (RBG) intron 2 fragment, and an Oryctolagus cuniculus β-globin exon 3 fragment.

The UTR may comprise a Kozak consensus sequence. Any suitable Kozak consensus sequence may be used, the selection of which may be readily made by the skilled person.

In a preferred embodiment, the UTR comprises the nucleic acid sequence specified in SEQ ID NO: 6 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The UTR of SEQ ID NO: 6 is 186 nucleotides in length and includes a Gallus gallus β-actin (CBA) intron 1 fragment (with predicted splice donor site), Oryctolagus cuniculus β-globin (RBG) intron 2 fragment (including predicted branch point and splice acceptor site) and Oryctolagus cuniculus β-globin exon 3 fragment immediately prior to a Kozak consensus sequence.

The present inventors have surprisingly found that the presence of a UTR as described above, in particular a UTR sequence as specified in SEQ ID NO: 6 or a variant thereof having at least 90% sequence identity, advantageously increases translational yield from the ABCA4 transgene.

The second (“downstream”) AAV vector of the AAV vector system of the invention may) comprise a post-transcriptional response element (also known as post-transcriptional regulatory element) or PRE. Any suitable PRE may be used, the selection of which may be readily made by the skilled person. The presence of a suitable PRE may enhance expression of the ABCA4 transgene.

In a preferred embodiment, the PRE is a Woodchuck Hepatitis Virus PRE (WPRE). In a particularly preferred embodiment, the WPRE has a sequence as specified in SEQ ID NO: 7 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The second AAV vector may comprise a poly-adenylation sequence located 3′ to the downstream ABCA4 nucleic acid sequence. Any suitable poly-adenylation sequence may be used, the selection of which may be readily made by the skilled person.

In a preferred embodiment, the poly-adenylation sequence is a bovine Growth Hormone (bGH) poly-adenylation sequence. In a particularly preferred embodiment, the bGH poly-adenlylation sequence has a sequence as specified in SEQ ID NO: 8 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In a preferred embodiment of the AAV vector system of the invention, the first AAV vector comprises the nucleic acid sequence of SEQ ID NO: 9, and the second AAV vector comprises the nucleic acid sequence of SEQ ID NO: 10.

In another preferred embodiment of the AAV vector system of the invention, the first AAV vector comprises the nucleic acid sequence of SEQ ID NO: 3, and the second AAV vector comprises the nucleic acid sequence of SEQ ID NO: 4.

The AAV vector system of the invention is suitable for expressing a human ABCA4 protein in a target cell.

Thus, in one aspect, the invention provides a method for expressing a human ABCA4 protein in a target cell, the method comprising the steps of: transducing the target cell with the first AAV vector and the second AAV vector as described above, such that a functional ABCA4 protein is expressed in the target cell.

Expression of human ABCA4 protein requires that the target cell be transduced with both the first AAV vector and the second AAV vector; however, the order is not important. Thus, the target cell may be transduced with the first AAV vector and the second AAV vector in any order (first AAV vector followed by second AAV vector, or second AAV vector followed by first AAV vector) or simultaneously.

Methods for transducing target cells with AAV vectors are known in the art and will be familiar to a skilled person.

The target cell is preferably a cell of the eye, preferably a retinal cell (e.g. a neuronal photoreceptor cell, a rod cell, a cone cell, or a retinal pigment epithelium cell).

The present invention also provides the first AAV vector, as defined above. There is also provided the second AAV vector, as defined above.

In another aspect, the invention provides an AAV vector, comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1. Accordingly, this AAV vector does not comprise any additional ABCA4 CDS beyond said sequence of contiguous nucleotides.

The first AAV vector may comprise 5′ and 3′ ITRs, preferably AAV ITRs; a promoter, preferably a GRK1 promoter; and/or a UTR; said elements being as described above in relation to the AAV vector system of the invention.

In one embodiment, the first AAV vector comprises the nucleic acid sequence of SEQ ID NO: 9.

In one embodiment, the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the first AAV vector comprises the nucleic acid sequence of SEQ ID NO: 9 with the proviso that the nucleotide at the position corresponding to nucleotide 1640 of SEQ ID NO: 1 is G, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the first AAV vector comprises the nucleic acid sequence of SEQ ID NO: 3.

In one embodiment, the first AAV vector comprises the nucleic acid sequence of SEQ ID NO: 3 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the first AAV vector comprises the nucleic acid sequence of SEQ ID NO: 3 with the proviso that the nucleotide at the position corresponding to nucleotide 1640 of SEQ ID NO: 1 is G. or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In another aspect, the invention provides an AAV vector, comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1. Accordingly, this AAV vector does not comprise any additional ABCA4 CDS beyond said sequence of contiguous nucleotides.

The second vector may comprise 5′ and 3′ ITRs, preferably AAV ITRs; a PRE, preferably a WPRE; and/or a poly-adenylation sequence, preferably a bGH poly-adenylation sequence; said elements being as described above in relation to the AAV vector system of the invention.

In one embodiment, the second AAV vector comprises the nucleic acid sequence of SEQ ID NO: 10.

In one embodiment, the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the second AAV vector comprises the nucleic acid sequence of SEQ ID NO: 10 with the proviso that the nucleotide at the position corresponding to nucleotide 5279 of SEQ ID NO: 1 is G and the nucleotide at the position corresponding to nucleotide 6173 of SEQ ID NO: 1 is T, or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the second AAV vector comprises the nucleic acid sequence of SEQ ID NO: 4.

In one embodiment, the second AAV vector comprises the nucleic acid sequence of SEQ ID NO: 4 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

In one embodiment, the second AAV vector comprises the nucleic acid sequence of SEQ ID NO: 4 with the proviso that the nucleotide at the position corresponding to nucleotide 5279 of SEQ ID NO: 1 is G and the nucleotide at the position corresponding to nucleotide 6173 of SEQ ID NO: 1 is T. or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The invention also provides nucleic acids comprising the nucleic acid sequences described above.

The invention also provides an AAV vector genome derivable from an AAV vector as described above.

Also provided is a kit comprising the first AAV vector and the second AAV vector as described above. The AAV vectors may be provided in the kits in the form of AAV particles.

Further provided is a kit comprising a nucleic acid comprising the first nucleic acid sequence and a nucleic acid comprising the second nucleic acid sequence, as described above.

The invention also provides a pharmaceutical composition comprising the AAV vector system as described above and a pharmaceutically acceptable excipient.

The AAV vector system of the invention, the kit of the invention, and the pharmaceutical composition of the invention, may be used in gene therapy. For example, AAV vector system of the invention, the kit of the invention, and the pharmaceutical composition of the invention, may be used in preventing or treating disease.

Use of the present invention to prevent or treat disease requires administration of the first AAV vector and second AAV vector to a target cell, to provide expression of ABCA4 protein.

Preferably the disease to be prevented or treated is characterised by degradation of retinal cells. An example of such a disease is Stargardt disease. Accordingly, the first and second AAV vectors of the invention may be administered to an eye of a patient, preferably to retinal tissue of the eye, such that functional ABCA4 protein is expressed to compensate for the mutation(s) present in the disease.

The AAV vectors of the invention may be formulated as pharmaceutical compositions or medicaments.

An example AAV vector system of the invention comprises a first AAV vector and a second AAV vector; wherein the first AAV vector comprises the nucleic acid sequence of SEQ ID NO: 9; and the second AAV vector comprises the nucleic acid sequence of SEQ ID NO: 10.

A further example AAV vector system of the invention comprises a first AAV vector and a second AAV vector; wherein the first AAV vector comprises the nucleic acid sequence of SEQ ID NO: 9 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity; and the second AAV vector comprises the nucleic acid sequence of SEQ ID NO: 10 or a variant thereof having at least 90% (e.g. at least 90, 95, 96, 97, 98, 99, 99.1, 99.2, 99.3, 99.4, 99.5, 99.6, 99.7, 99.8 or 99.9%) sequence identity.

The present invention may also be performed where SEQ ID NO: 2 is used as a reference sequence in place of SEQ ID NO: 1.

In this regard, SEQ ID NO: 2 is identical to SEQ ID NO: 1 with the exception of the following mutations: nucleotide 1640 G>T, nucleotide 5279 G>A, nucleotide 6173 T>C.

These mutations do not alter the encoded amino acid sequence, and thus the ABCA4 protein encoded by SEQ ID NO: 2 is identical to the ABCA4 protein encoded by SEQ ID NO: 1.

Thus, in alternative embodiments of the invention, references above to SEQ ID NO: 1 may be replaced with references to SEQ ID NO: 2.

### Sequence Correspondence

As used herein, the term “corresponding to” when used with regard to the nucleotides in a given nucleic acid sequence defines nucleotide positions by reference to a particular SEQ ID NO. However, when such references are made, it will be understood that the invention is not to be limited to the exact sequence as set out in the particular SEQ ID NO referred to but includes variant sequences thereof. The nucleotides corresponding to the nucleotide positions in SEQ ID NO: 1 can be readily determined by sequence alignment, such as by using sequence alignment programs, the use of which is well known in the art. In this regard, a skilled person would readily appreciate that the degenerate nature of the genetic code means that variations in a nucleic acid sequence encoding a given polypeptide may be present without changing the amino acid sequence of the encoded polypeptide. Thus, identification of nucleotide locations in other ABCA4 coding sequences is contemplated (i.e. nucleotides at positions which the skilled person would consider correspond to the positions identified in, for example, SEQ ID NO: 1).

By way of example, SEQ ID NO: 2 is identical to SEQ ID NO: 1 with the exception of three specific mutations, as described above (these three mutations do not alter the amino acid sequence of the encoded ABCA4 polypeptide). In this case, a skilled person would therefore consider that a given nucleotide position in SEQ ID NO: 2 corresponded to the equivalent numbered nucleotide position in SEQ ID NO: 1.

### AAV Vectors

The viral vectors of the invention are adeno-associated viral (AAV) vectors. An AAV vector of the invention may be in the form of a mature AAV particle or virion, i.e. nucleic acid surrounded by an AAV protein capsid.

The AAV vector may comprise an AAV genome or a derivative thereof.

An AAV genome is a polynucleotide sequence, which encodes functions needed for production of an AAV particle. These functions include those operating in the replication and packaging cycle of AAV in a host cell, including encapsidation of the AAV genome into an AAV particle. Naturally occurring AAVs are replication-deficient and rely on the provision of helper functions in trans for completion of a replication and packaging cycle. Accordingly, an AAV genome of a vector of the invention is typically replication-deficient.

The AAV genome may be in single-stranded form, either positive or negative-sense, or alternatively in double-stranded form. The use of a double-stranded form allows bypass of the DNA replication step in the target cell and so can accelerate transgene expression.

The AAV genome of a vector of the invention is typically in single-stranded form.

The AAV genome may be from any naturally derived serotype, isolate or clade of AAV. Thus, the AAV genome may be the full genome of a naturally occurring AAV. As is known to the skilled person, AAVs occurring in nature may be classified according to various biological systems.

Commonly, AAVs are referred to in terms of their serotype. A serotype corresponds to a variant subspecies of AAV which, owing to its profile of expression of capsid surface antigens, has a distinctive reactivity which can be used to distinguish it from other variant subspecies. Typically, a virus having a particular AAV serotype does not efficiently cross-react with neutralising antibodies specific for any other AAV serotype.

AAV serotypes include AAV1, AAV2, AAV3, AAV4, AAV5, AAV6, AAV7, AAV8, AAV9, AAV10 and AAV11, and also recombinant serotypes, such as Rec2 and Rec3, recently identified from primate brain. Any of these AAV serotypes may be used in the invention. Thus, in one embodiment of the invention, an AAV vector of the invention may be derived from an AAV1, AAV2, AAV3, AAV4, AAV5, AAV6, AAV7, AAV8, AAV9, AAV10, AAV11. Rec2 or Rec3 AAV.

Reviews of AAV serotypes may be found in Choi et al. (2005) Curr. Gene Ther. 5: 299-310 and Wu et al. (2006) Molecular Therapy 14: 316-27. The sequences of AAV genomes or of elements of AAV genomes including ITR sequences, rep or cap genes may be derived from the following accession numbers for AAV whole genome sequences: Adeno-associated virus 1 NC_002077, AF063497; Adeno-associated virus 2 NC_001401; Adeno-associated virus 3 NC_001729; Adeno-associated virus 3B NC_001863; Adeno-associated virus 4 NC_001829; Adeno-associated virus 5 Y18065, AF085716; Adeno-associated virus 6 NC_001862; Avian AAV ATCC VR-865 AY186198, AY629583, NC_004828; Avian AAV strain DA-1 NC_006263, AY629583; Bovine AAV NC_005889, AY388617.

AAV may also be referred to in terms of clades or clones. This refers to the phylogenetic relationship of naturally derived AAVs, and typically to a phylogenetic group of AAVs which can be traced back to a common ancestor, and includes all descendants thereof.

Additionally, AAVs may be referred to in terms of a specific isolate, i.e. a genetic isolate of a specific AAV found in nature. The term genetic isolate describes a population of AAVs which has undergone limited genetic mixing with other naturally occurring AAVs, thereby defining a recognisably distinct population at a genetic level.

The skilled person can select an appropriate serotype, clade, clone or isolate of AAV for use in the invention on the basis of their common general knowledge. For instance, the AAV5 capsid has been shown to transduce primate cone photoreceptors efficiently as evidenced by the successful correction of an inherited colour vision defect (Mancuso et al. (2009) Nature 461: 784-7).

The AAV serotype determines the tissue specificity of infection (or tropism) of an AAV virus. Accordingly, preferred AAV serotypes for use in AAVs administered to patients in accordance with the invention are those which have natural tropism for or a high efficiency of infection of target cells within the eye. In one embodiment, AAV serotypes for use in the invention are those which infect cells of the neurosensory retina, retinal pigment epithelium and/or choroid.

Typically, the AAV genome of a naturally derived serotype, isolate or clade of AAV comprises at least one inverted terminal repeat sequence (ITR). An ITR sequence acts in cis to provide a functional origin of replication and allows for integration and excision of the vector from the genome of a cell. The AAV genome typically also comprises packaging genes, such as rep and/or cap genes which encode packaging functions for an AAV particle. The rep gene encodes one or more of the proteins Rep78, Rep68, Rep52 and Rep40 or variants thereof. The cap gene encodes one or more capsid proteins such as VP1, VP2 and VP3 or variants thereof. These proteins make up the capsid of an AAV particle. Capsid variants are discussed below.

A promoter will be operably linked to each of the packaging genes. Specific examples of such promoters include the p5, p19 and p40 promoters (Laughlin et al. (1979) Proc. Natl. Acad. Sci. USA 76: 5567-5571). For example, the p5 and p19 promoters are generally used to express the rep gene, while the p40 promoter is generally used to express the cap gene.

The AAV genome used in a vector of the invention may therefore be the full genome of a naturally occurring AAV. For example, a vector comprising a full AAV genome may be used to prepare an AAV vector in vitro. However, while such a vector may in principle be administered to patients, this will rarely be done in practice. Preferably the AAV genome will be derivatised for the purpose of administration to patients. Such derivatisation is standard in the art and the invention encompasses the use of any known derivative of an AAV genome, and derivatives which could be generated by applying techniques known in the art. Derivatisation of the AAV genome and of the AAV capsid are reviewed in Coura and Nardi (2007) Virology Journal 4: 99, and in Choi et al. and Wu et al., referenced above.

Derivatives of an AAV genome include any truncated or modified forms of an AAV genome which allow for expression of a transgene from a vector of the invention in vivo. Typically, it is possible to truncate the AAV genome significantly to include minimal viral sequence yet retain the above function. This is preferred for safety reasons to reduce the risk of recombination of the vector with wild-type virus, and also to avoid triggering a cellular immune response by the presence of viral gene proteins in the target cell.

Typically, a derivative of an AAV genome will include at least one inverted terminal repeat sequence (ITR), preferably more than one ITR, such as two ITRs or more. One or more of the ITRs may be derived from AAV genomes having different serotypes, or may be a chimeric or mutant ITR. A preferred mutant ITR is one having a deletion of a trs (terminal resolution site). This deletion allows for continued replication of the genome to generate a single-stranded genome which contains both coding and complementary sequences, i.e. a self-complementary AAV genome. This allows for bypass of DNA replication in the target cell, and so enables accelerated transgene expression.

The inclusion of one or more ITRs is preferred to aid concatamer formation of a vector of the invention in the nucleus of a host cell, for example following the conversion of single-stranded vector DNA into double-stranded DNA by the action of host cell DNA polymerases. The formation of such episomal concatamers protects the vector construct during the life of the host cell, thereby allowing for prolonged expression of the transgene in vivo.

In preferred embodiments, ITR elements will be the only sequences retained from the native AAV genome in the derivative. Thus, a derivative will preferably not include the rep and/or cap genes of the native genome and any other sequences of the native genome. This is preferred for the reasons described above, and also to reduce the possibility of integration of the vector into the host cell genome. Additionally, reducing the size of the AAV genome allows for increased flexibility in incorporating other sequence elements (such as regulatory elements) within the vector in addition to the transgene.

The following portions could therefore be removed in a derivative of the invention: one inverted terminal repeat (ITR) sequence, the replication (rep) and capsid (cap) genes. However, in some embodiments, derivatives may additionally include one or more rep and/or cap genes or other viral sequences of an AAV genome. Naturally occurring AAV integrates with a high frequency at a specific site on human chromosome 19, and shows a negligible frequency of random integration, such that retention of an integrative capacity in the vector may be tolerated in a therapeutic setting.

Where a derivative comprises capsid proteins i.e. VP1, VP2 and/or VP3, the derivative may be a chimeric, shuffled or capsid-modified derivative of one or more naturally occurring AAVs. In particular, the invention encompasses the provision of capsid protein sequences from different serotypes, clades, clones, or isolates of AAV within the same vector (i.e. a pseudotyped vector).

Chimeric, shuffled or capsid-modified derivatives will be typically selected to provide one or more desired functionalities for the viral vector. Thus, these derivatives may display increased efficiency of gene delivery, decreased immunogenicity (humoral or cellular), an altered tropism range and/or improved targeting of a particular cell type compared to an AAV vector comprising a naturally occurring AAV genome, such as that of AAV2. Increased efficiency of gene delivery may be effected by improved receptor or co-receptor binding at the cell surface, improved internalisation, improved trafficking within the cell and into the nucleus, improved uncoating of the viral particle and improved conversion of a single-stranded genome to double-stranded form Increased efficiency may also relate to an altered tropism range or targeting of a specific cell population, such that the vector dose is not diluted by administration to tissues where it is not needed.

Chimeric capsid proteins include those generated by recombination between two or more capsid coding sequences of naturally occurring AAV serotypes. This may be performed for example by a marker rescue approach in which non-infectious capsid sequences of one serotype are co-transfected with capsid sequences of a different serotype, and directed selection is used to select for capsid sequences having desired properties. The capsid sequences of the different serotypes can be altered by homologous recombination within the cell to produce novel chimeric capsid proteins.

Chimeric capsid proteins also include those generated by engineering of capsid protein sequences to transfer specific capsid protein domains, surface loops or specific amino acid residues between two or more capsid proteins, for example between two or more capsid proteins of different serotypes.

Shuffled or chimeric capsid proteins may also be generated by DNA shuffling or by error-prone PCR. Hybrid AAV capsid genes can be created by randomly fragmenting the sequences of related AAV genes e.g. those encoding capsid proteins of multiple different serotypes and then subsequently reassembling the fragments in a self-priming polymerase reaction, which may also cause crossovers in regions of sequence homology. A library of hybrid AAV genes created in this way by shuffling the capsid genes of several serotypes can be screened to identify viral clones having a desired functionality. Similarly, error prone PCR may be used to randomly mutate AAV capsid genes to create a diverse library of variants which may then be selected for a desired property.

The sequences of the capsid genes may also be genetically modified to introduce specific deletions, substitutions or insertions with respect to the native wild-type sequence. In particular, capsid genes may be modified by the insertion of a sequence of an unrelated protein or peptide within an open reading frame of a capsid coding sequence, or at the N- and/or C-terminus of a capsid coding sequence.

The unrelated protein or peptide may advantageously be one which acts as a ligand for a particular cell type, thereby conferring improved binding to a target cell or improving the specificity of targeting of the vector to a particular cell population. The unrelated protein may also be one which assists purification of the viral particle as part of the production process, i.e. an epitope or affinity tag. The site of insertion will typically be selected so as not to interfere with other functions of the viral particle e.g. internalisation, trafficking of the viral particle. The skilled person can identify suitable sites for insertion based on their common general knowledge. Particular sites are disclosed in Choi et al., referenced above.

The invention additionally encompasses the provision of sequences of an AAV genome in a different order and configuration to that of a native AAV genome. The invention also encompasses the replacement of one or more AAV sequences or genes with sequences from another virus or with chimeric genes composed of sequences from more than one virus. Such chimeric genes may be composed of sequences from two or more related viral proteins of different viral species.

AAV vectors of the invention include transcapsidated forms wherein an AAV genome or derivative having an ITR of one serotype is packaged in the capsid of a different serotype. AAV vectors of the invention also include mosaic forms wherein a mixture of unmodified capsid proteins from two or more different serotypes makes up the viral capsid. An AAV vector may also include chemically modified forms bearing ligands adsorbed to the capsid surface. For example, such ligands may include antibodies for targeting a particular cell surface receptor.

Thus, for example, AAV vectors of the invention include those with an AAV2 genome and AAV2 capsid proteins (AAV2/2), those with an AAV2 genome and AAV5 capsid proteins (AAV2/5) and those with an AAV2 genome and AAV8 capsid proteins (AAV2′8).

An AAV vector of the invention may comprise a mutant AAV capsid protein. In one embodiment, an AAV vector of the invention comprises a mutant AAV8 capsid protein. Preferably the mutant AAV8 capsid protein is an AAV8 Y733F capsid protein.

### Methods of Administration

The viral vectors of the invention may be administered to the eye of a subject by subretinal, direct retinal or intravitreal injection.

A skilled person will be familiar with and well able to carry out individual subretinal, direct retinal or intravitreal injections.

### Subretinal Injection

Subretinal injections are injections into the subretinal space, i.e. underneath the neurosensory retina. During a subretinal injection, the injected material is directed into, and creates a space between, the photoreceptor cell and retinal pigment epithelial (RPE) layers.

When the injection is carried out through a small retinotomy, a retinal detachment may be created. The detached, raised layer of the retina that is generated by the injected material is referred to as a “bleb”.

The hole created by the subretinal injection must be sufficiently small that the injected solution does not significantly reflux back into the vitreous cavity after administration. Such reflux would be particularly problematic when a medicament is injected, because the effects of the medicament would be directed away from the target zone. Preferably, the injection creates a self-sealing entry point in the neurosensory retina, i.e. once the injection needle is removed, the hole created by the needle reseals such that very little or substantially no injected material is released through the hole.

To facilitate this process, specialist subretinal injection needles are commercially available (e.g. DORC 41G Teflon subretinal injection needle, Dutch Ophthalmic Research Center International BV, Zuidland, The Netherlands). These are needles designed to carry out subretinal injections.

Unless damage to the retina occurs during the injection, and as long as a sufficiently small needle is used, substantially all injected material remains localised between the detached neurosensory retina and the RPE at the site of the localised retinal detachment (i.e. does not reflux into the vitreous cavity). Indeed, the typical persistence of the bleb over a short time frame indicates that there is usually little escape of the injected material into the vitreous. The bleb may dissipate over a longer time frame as the injected material is absorbed.

Visualisations of the eye, in particular the retina, for example using optical coherence tomography, may be made pre-operatively.

### Two-Step Subretinal Injection

The AAV vectors of the invention may be delivered with increased accuracy and safety by using a two-step method in which a localised retinal detachment is created by the subretinal injection of a first solution. The first solution does not comprise the vector. A second subretinal injection is then used to deliver the medicament comprising the vector into the subretinal fluid of the bleb created by the first subretinal injection. Because the injection delivering the medicament is not being used to detach the retina, a specific volume of solution may be injected in this second step.

An AAV vector of the invention may be delivered by:

(a) administering a solution to the subject by subretinal injection in an amount effective to at least partially detach the retina to form a subretinal bleb, wherein the solution does not comprise the vector; and
(b) administering a medicament composition by subretinal injection into the bleb formed by step (a), wherein the medicament comprises the vector.

The volume of solution injected in step (a) to at least partially detach the retina may be, for example, about 10-1000 μL, for example about 50-1000, 100-1000, 250-1000, 500-1000, 10-500, 50-500, 100-500, 250-500 μL. The volume may be, for example, about 10, 50, 100, 200, 300, 400, 500, 600, 700, 800, 900 or 1000 μL.

The volume of the medicament composition injected in step (b) may be, for example, about 10-500 μL, for example about 50-500, 100-500, 200-500, 300-500, 400-500, 50-250, 100-250, 200-250 or 50-150 μL. The volume may be, for example, about 10, 50, 100, 150, 200, 250, 300, 350, 400, 450 or 500 μL. Preferably, the volume of the medicament composition injected in step (b) is 100 μL. Larger volumes may increase the risk of stretching the retina, while smaller volumes may be difficult to see.

The solution that does not comprise the medicament (i.e. the “first solution” of step (a)) may be similarly formulated to the solution that does comprise the medicament, as described below. A preferred solution that does not comprise the medicament is balanced saline solution (BSS) or a similar buffer solution matched to the pH and osmolality of the subretinal space.

### Visualising the Retina During Surgery

Under certain circumstances, for example during end-stage retinal degenerations, identifying the retina is difficult because it is thin, transparent and difficult to see against the disrupted and heavily pigmented epithelium on which it sits. The use of a blue vital dye (e.g. Brilliant Peel®, Geuder; MembraneBlue-Dual®, Dorc) may facilitate the identification of the retinal hole made for the retinal detachment procedure (i.e. step (a) in the two-step subretinal injection method of the invention) so that the medicament can be administered through the same hole without the risk of reflux back into the vitreous cavity.

The use of the blue vital dye also identifies any regions of the retina where there is a thickened internal limiting membrane or epiretinal membrane, as injection through either of these structures would hinder clean access into the subretinal space. Furthermore, contraction of either of these structures in the immediate post-operative period could lead to stretching of the retinal entry hole, which could lead to reflux of the medicament into the vitreous cavity.

### Pharmaceutical Compositions and Injected Solutions

The AAV vectors and AAV vector system of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, e.g. subretinal, direct retinal or intravitreal injection.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used.

For injection at the site of affliction, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection, Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

### Method of Treatment

It is to be appreciated that all references herein to treatment include curative, palliative and prophylactic treatment; although in the context of the invention references to preventing are more commonly associated with prophylactic treatment. Treatment may also include arresting progression in the severity of a disease.

The treatment of mammals, particularly humans, is preferred. However, both human and veterinary treatments are within the scope of the invention.

### Variants, Derivatives, Analogues, Homologues and Fragments

In addition to the specific proteins and nucleotides mentioned herein, the invention also encompasses the use of variants, derivatives, analogues, homologues and fragments thereof.

In the context of the invention, a variant of any given sequence is a sequence in which the specific sequence of residues (whether amino acid or nucleic acid residues) has been modified in such a manner that the polypeptide or polynucleotide in question substantially retains its function. A variant sequence can be obtained by addition, deletion, substitution, modification, replacement and/or variation of at least one residue present in the naturally-occurring protein.

The term “derivative” as used herein, in relation to proteins or polypeptides of the invention includes any substitution of, variation of, modification of, replacement of, deletion of and/or addition of one (or more) amino acid residues from or to the sequence providing that the resultant protein or polypeptide substantially retains at least one of its endogenous functions.

The term “analogue” as used herein, in relation to polypeptides or polynucleotides includes any mimetic, that is, a chemical compound that possesses at least one of the endogenous functions of the polypeptides or polynucleotides which it mimics.

Typically, amino acid substitutions may be made, for example from 1, 2 or 3 to 10 or 20 substitutions provided that the modified sequence substantially retains the required activity or ability. Amino acid substitutions may include the use of non-naturally occurring analogues. Proteins used in the invention may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to the table below. Amino acids in the same block in the second column and preferably in the same line in the third column may be substituted for each other:

| | | |
|---|---|---|
| ALIPHATIC | Non-polar | G A P |
| | | I L V |
| | Polar—uncharged | C S T M |
| | | N Q |
| | Polar—charged | D E |
| | | K R H |
| AROMATIC | | F W Y |

The term “homologue” as used herein means an entity having a certain homology with the wild type amino acid sequence and the wild type nucleotide sequence. The term “homology” can be equated with “identity”.

A homologous sequence may include an amino acid sequence which may be at least 50%, 55%, 60%, 65%, 70%, 75%, 80%, 85% or 90% identical, preferably at least 95% or 97% or 99% identical to the subject sequence. Typically, the homologues will comprise the same active sites etc. as the subject amino acid sequence. Although homology can also be considered in terms of similarity (i.e. amino acid residues having similar chemical properties/functions), in the context of the invention it is preferred to express homology in terms of sequence identity.

A homologous sequence may include a nucleotide sequence which may be at least 50%, 55%, 60%, 65%, 70%, 75%, 80%, 85% or 90% identical, preferably at least 95% or 97% or 99% identical to the subject sequence. Although homology can also be considered in terms of similarity, in the context of the invention it is preferred to express homology in terms of sequence identity.

Preferably, reference to a sequence which has a percent identity to any one of the SEQ ID NOs detailed herein refers to a sequence which has the stated percent identity over the entire length of the SEQ ID NO referred to.

Homology comparisons can be conducted by eye or, more usually, with the aid of readily available sequence comparison programs. These commercially available computer programs can calculate percentage homology or identity between two or more sequences.

Percentage homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum percentage homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12: 387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid. pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174: 247-50; FEMS Microbiol. Lett. (1999) 177: 187-8). Although the final percent homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62.

Once the software has produced an optimal alignment, it is possible to calculate percent homology, preferably percent sequence identity. The software typically does this as part of the sequence comparison and generates a numerical result.

“Fragments” are also variants and the term typically refers to a selected region of the polypeptide or polynucleotide that is of interest either functionally or, for example, in an assay. “Fragment” thus refers to an amino acid or nucleic acid sequence that is a portion of a full-length polypeptide or polynucleotide.

Such variants may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used

### Codon Optimisation

The present invention encompasses codon optimised variants of the nucleic acid sequences described herein.

Codon optimisation takes advantage of redundancies in the genetic code to enable a nucleotide sequence to be altered while maintaining the same amino acid sequence of the encoded protein.

Typically, codon optimisation is carried out to facilitate an increase or decrease in the expression of an encoded protein. This is effected by tailoring codon usage in a nucleotide sequence to that of a specific cell type, thus taking advantage of cellular codon bias corresponding to a bias in the relative abundance of particular tRNAs in the cell type. By altering the codons in the nucleotide sequence so that they are tailored to match the relative abundance of corresponding tRNAs, it is possible to increase expression. Conversely, it is possible to decrease expression by selecting codons for which the corresponding tRNAs are known to be rare in the particular cell type.

Methods for codon optimisation of nucleic acid sequences are known in the art and will be familiar to a skilled person.

SEQUENCES

SEQ ID NO: 1
AGGACACAGCGTCCGGAGCCAGAGGCGCTCTTAACGGCGTTTATGTCCTTTGCTGTCTGAGGGGCCTCAGCTCTG
ACCAATCTGGTCTTCGTGTGGTCATTAGCATGGGCTTCGTGAGACAGATACAGCTTTTGCTCTGGAAGAACTGGA
CCCTGCGGAAAAGGCAAAAGATTCGCTTTGTGGTGGAACTCGTGTGGCCTTTATCTTTATTTCTGGTCTTGATCT
GGTTAAGGAATGCCAACCCGCTCTACAGCCATCATGAATGCCATTTCCCCAACAAGGCGATGCCCTCAGCAGGAA
TGCTGCCGTGGCTCCAGGGGATCTTCTGCAATGTGAACAATCCCTGTTTTCAAAGCCCCACCCCAGGAGAATCTC
CTGGAATTGTGTCAAACTATAACAACTCCATCTTGGCAAGGGTATATCGAGATTTTCAAGAACTCCTCATGAATG
CACCAGAGAGCCAGCACCTTGGCCGTATTTGGACAGAGCTACACATCTTGTCCCAATTCATGGACACCCTCCGGA
CTCACCCGGAGAGAATTGCAGGAAGAGGAATACGAATAAGGGATATCTTGAAAGATGAAGAAACACTGACACTAT
TTCTCATTAAAAACATCGGCCTGTCTGACTCAGTGGTCTACCTTCTGATCAACTCTCAAGTCCGTCCAGAGCAGT
TCGCTCATGGAGTCCCGGACCTGGCGCTGAAGGACATCGCCTGCAGCGAGGCCCTCCTGGAGCGCTTCATCATCT
TCAGCCAGAGACGCGGGGCAAAGACGGTGCGCTATGCCCTGTGCTCCCTCTCCCAGGGCACCCTACAGTGGATAG
AAGACACTCTGTATGCCAACGTGGACTTCTTCAAGCTCTTCCGTGTGCTTCCCACACTCCTAGACAGCCGTTCTC
AAGGTATCAATCTGAGATCTTGGGGAGGAATATTATCTGATATGTCACCAAGAATTCAAGAGTTTATCCATCGGC
CGAGTATGCAGGACTTGCTGTGGGTGACCAGGCCCCTCATGCAGAATGGTGGTCCAGAGACCTTTACAAAGCTGA
TGGGCATCCTGTCTGACCTCCTGTGTGGCTACCCCGAGGGAGGTGGCTCTCGGGTGCTCTCCTTCAACTGGTATG
AAGACAATAACTATAAGGCCTTTCTGGGGATTGACTCCACAAGGAAGGATCCTATCTATTCTTATGACAGAAGAA
CAACATCCTTTTGTAATGCATTGATCCAGAGCCTGGAGTCAAATCCTTTAACCAAAATCGCTTGGAGGGCGGCAA
AGCCTTTGCTGATGGGAAAAATCCTGTACACTCCTGATTCACCTGCAGCACGAAGGATACTGAAGAATGCCAACT
CAACTTTTGAAGAACTGGAACACGTTAGGAAGTTGGTCAAAGCCTGGGAAGAAGTAGGGCCCCAGATCTGGTACT
TCTTTGACAACAGCACACAGATGAACATGATCAGAGATACCCTGGGGAACCCAACAGTAAAAGACTTTTTGAATA
GGCAGCTTGGTGAAGAAGGTATTACTGCTGAAGCCATCCTAAACTTCCTCTACAAGGGCCCTCGGGAAAGCCAGG
CTGACGACATGGCCAACTTCGACTGGAGGGACATATTTAACATCACTGATCGCACCCTCCGCCTGGTCAATCAAT
ACCTGGAGTGCTTGGTCCTGGATAAGTTTGAAAGCTACAATGATGAAACTCAGCTCACCCAACGTGCCCTCTCTC
TACTGGAGGAAAACATGTTCTGGGCCGGAGTGGTATTCCCTGACATGTATCCCTGGACCAGCTCTCTACCACCCC
ACGTGAAGTATAAGATCCGAATGGACATAGACGTGGTGGAGAAAACCAATAAGATTAAAGACAGGTATTGGGATT
CTGGTCCCAGAGCTGATCCCGTGGAAGATTTCCGGTACATCTGGGGCGGGTTTGCCTATCTGCAGGACATGGTTG
AACAGGGGATCACAAGGAGCCAGGTGCAGGCGGAGGCTCCAGTTGGAATCTACCTCCAGCAGATGCCCTACCCCT
GCTTCGTGGACGATTCTTTCATGATCATCCTGAACCGCTGTTTCCCTATCTTCATGGTGCTGGCATGGATCTACT
CTGTCTCCATGACTGTGAAGAGCATCGTCTTGGAGAAGGAGTTGCGACTGAAGGAGACCTTGAAAAATCAGGGTG
TCTCCAATGCAGTGATTTGGTGTACCTGGTTCCTGGACAGCTTCTCCATCATGTCGATGAGCATCTTCCTCCTGA
CGATATTCATCATGCATGGAAGAATCCTACATTACAGCGACCCATTCATCCTCTTCCTGTTCTTGTTGGCTTTCT
CCACTGCCACCATCATGCTGTGCTTTCTGCTCAGCACCTTCTTCTCCAAGGCCAGTCTGGCAGCAGCCTGTAGTG
GTGTCATCTATTTCACCCTCTACCTGCCACACATCCTGTGCTTCGCCTGGCAGGACCGCATGACCGCTGAGCTGA
AGAAGGCTGTGAGCTTACTGTCTCCGGTGGCATTTGGATTTGGCACTGAGTACCTGGTTCGCTTTGAAGAGCAAG
GCCTGGGGCTGCAGTGGAGCAACATCGGGAACAGTCCCACGGAAGGGGACGAATTCAGCTTCCTGCTGTCCATGC
AGATGATGCTCCTTGATGCTGCTGTCTATGGCTTACTCGCTTGGTACCTTGATCAGGTGTTTCCAGGAGACTATG
GAACCCCACTTCCTTGGTACTTTCTTCTACAAGAGTCGTATTGGCTTGGCGGTGAAGGGTGTTCAACCAGAGAAG
AAAGAGCCCTGGAAAAGACCGAGCCCCTAACAGAGGAAACGGAGGATCCAGAGCACCCAGAAGGAATACACGACT
CCTTCTTTGAACGTGAGCATCCAGGGTGGGTTCCTGGGGTATGCGTGAAGAATCTGGTAAAGATTTTTGAGCCCT
GTGGCCGGCCAGCTGTGGACCGTCTGAACATCACCTTCTACGAGAACCAGATCACCGCATTCCTGGGCCACAATG
GAGCTGGGAAAACCACCACCTTGTCCATCCTGACGGGTCTGTTGCCACCAACCTCTGGGACTGTGCTCGTTGGGG
GAAGGGACATTGAAACCAGCCTGGATGCAGTCCGGCAGAGCCTTGGCATGTGTCCACAGCACAACATCCTGTTCC
ACCACCTCACGGTGGCTGAGCACATGCTGTTCTATGCCCAGCTGAAAGGAAAGTCCCAGGAGGAGGCCCAGCTGG
AGATGGAAGCCATGTTGGAGGACACAGGCCTCCACCACAAGCGGAATGAAGAGGCTCAGGACCTATCAGGTGGCA
TGCAGAGAAAGCTGTCGGTTGCCATTGCCTTTGTGGGAGATGCCAAGGTGGTGATTCTGGACGAACCCACCTCTG
GGGTGGACCCTTACTCGAGACGCTCAATCTGGGATCTGCTCCTGAAGTATCGCTCAGGCAGAACCATCATCATGT
CCACTCACCACATGGACGAGGCCGACCTCCTTGGGGACCGCATTGCCATCATTGCCCAGGGAAGGCTCTACTGCT
CAGGCACCCCACTCTTCCTGAAGAACTGCTTTGGCACAGGCTTGTACTTAACCTTGGTGCGCAAGATGAAAAACA
TCCAGAGCCAAAGGAAAGGCAGTGAGGGGACCTGCAGCTGCTCGTCTAAGGGTTTCTCCACCACGTGTCCAGCCC
ACGTCGATGACCTAACTCCAGAACAAGTCCTGGATGGGGATGTAAATGAGCTGATGGATGTAGTTCTCCACCATG
TTCCAGAGGCAAAGCTGGTGGAGTGCATTGGTCAAGAACTTATCTTCCTTCTTCCAAATAAGAACTTCAAGCACA
GAGCATATGCCAGCCTTTTCAGAGAGCTGGAGGAGACGCTGGCTGACCTTGGTCTCAGCAGTTTTGGAATTTCTG
ACACTCCCCTGGAAGAGATTTTTCTGAAGGTCACGGAGGATTCTGATTCAGGACCTCTGTTTGCGGGTGGCGCTC
AGCAGAAAAGAGAAAACGTCAACCCCCGACACCCCTGCTTGGGTCCCAGAGAGAAGGCTGGACAGACACCCCAGG
ACTCCAATGTCTGCTCCCCAGGGGCGCCGGCTGCTCACCCAGAGGGCCAGCCTCCCCCAGAGCCAGAGTGCCCAG
GCCCGCAGCTCAACACGGGGACACAGCTGGTCCTCCAGCATGTGCAGGCGCTGCTGGTCAAGAGATTCCAACACA
CCATCCGCAGCCACAAGGACTTCCTGGCGCAGATCGTGCTCCCGGCTACCTTTGTGTTTTTGGCTCTGATGCTTT
CTATTGTTATCCCTCCTTTTGGCGAATACCCCGCTTTGACCCTTCACCCCTGGATATATGGGCAGCAGTACACCT
TCTTCAGCATGGATGAACCAGGCAGTGAGCAGTTCACGGTACTTGCAGACGTCCTCCTGAATAAGCCAGGCTTTG
GCAACCGCTGCCTGAAGGAAGGGTGGCTTCCGGAGTACCCCTGTGGCAACTCAACACCCTGGAAGACTCCTTCTG
TGTCCCCAAACATCACCCAGCTGTTCCAGAAGCAGAAATGGACACAGGTCAACCCTTCACCATCCTGCAGGTGCA
GCACCAGGGAGAAGCTCACCATGCTGCCAGAGTGCCCCGAGGGTGCCGGGGGCCTCCCGCCCCCCCAGAGAACAC
AGCGCAGCACGGAAATTCTACAAGACCTGACGGACAGGAACATCTCCGACTTCTTGGTAAAAACGTATCCTGCTC
TTATAAGAAGCAGCTTAAAGAGCAAATTCTGGGTCAATGAACAGAGGTATGGAGGAATTTCCATTGGAGGAAAGC
TCCCAGTCGTCCCCATCACGGGGGAAGCACTTGTTGGGTTTTTAAGCGACCTTGGCCGGATCATGAATGTGAGCG
GGGGCCCTATCACTAGAGAGGCCTCTAAAGAAATACCTGATTTCCTTAAACATCTAGAAACTGAAGACAACATTA
AGGTGTGGTTTAATAACAAAGGCTGGCQTGCCCTGGTCAGCTTTCTCAATGTGGCCCACAACGCCATCTTACGGG
CCAGCCTGCCTAAGGACAGGAGCCCCGAGGAGTATGGAATCACCGTCATTAGCCAACCCCTGAACCTGACCAAGG
AGCAGCTCTCAGAGATTACAGTGCTGACCACTTCAGTGGATGCTGTGGTTGCCATCTGCGTGATTTTCTCCATGT
CCTTCGTCCCAGCCAGCTTTGTCCTTTATTTGATCCAGGAGCGGGTGAACAAATCCAAGCACCTCCAGTTTATCA
GTGGAGTGAGCCCCACCACCTACTGGGTGACCAACTTCCTCTGGGACATCATGAATTATTCCGTGAGTGCTGGGC
TGGTGGTGGGCATCTTCATCGGGTTTCAGAAGAAAGCCTACACTTCTCCAGAAAACCTTCCTGCCCTTGTGGCAC
TGCTCCTGCTGTATGGATGGGCGGTCATTCCCATGATGTACCCAGCATCCTTCCTGTTTGATGTCCCCAGCACAG
CCTATGTGGCTTTATCTTGTGCTAATCTGTTCATCGGCATCAACAGCAGTGCTATTACCTTCATCTTGGAATTAT
TTGAGAATAACCGGACGCTGCTCAGGTTCAACGCCGTGCTGAGGAAGCTGCTCATTGTCTTCCCCCACTTCTGCC
TGGGCCGGGGCCTCATTGACCTTGCACTGAGCCAGGCTGTGACAGATGTCTATGCCCGGTTTGGTGAGGAGCACT
CTGCAAATCCGTTCCACTGGGACCTGATTGGGAAGAACCTGTTTGCCATGGTGGTGGAAGGGGTGGTGTACTTCC
TCCTGACCCTGCTGGTCCAGCGCCACTTCTTCCTCTCCCAATGGATTGCCGAGCCCACTAAGGAGCCCATTGTTG
ATGAAGATGATGATGTGGCTGAAGAAAGACAAAGAATTATTACTGGTGGAAATAAAACTGACATCTTAAGGCTAC
ATGAACTAACCAAGATTTATCCAGGCACCTCCAGCCCAGCAGTGGACAGGCTGTGTGTCGGAGTTCGCCCTGGAG
AGTGCTTTGGCCTCCTGGGAGTGAATGGTGCCGGCAAAACAACCACATTCAAGATGCTCACTGGGGACACCACAG
TGACCTCAGGGGATGCCACCGTAGCAGGCAAGAGTATTTTAACCAATATTTCTGAAGTCCATCAAAATATGGGCT
ACTGTCCTCAGTTTGATGCAATTGATGAGCTGCTCACAGGACGAGAACATCTTTACCTTTATGCCCGGCTTCGAG
GTGTACCAGCAGAAGAAATCGAAAAGGTTGCAAACTGGAGTATTAAGAGCCTGGGCCTGACTGTCTACGCCGACT
GCCTGGCTGGCACGTACAGTGGGGGCAACAAGCGGAAACTCTCCACAGCCATCGCACTCATTGGCTGCCCACCGC
TGGTGCTGCTGGATGAGCCCACCACAGGGATGGACCCCCAGGCACGCCGCATGCTGTGGAACGTCATCGTGAGCA
TCATCAGAGAAGGGAGGGCTGTGGTCCTCACATCCCACAGCATGGAAGAATGTGAGGCACTGTGTACCCGGCTGG
CCATCATGGTAAAGGGCGCCTTTCGATGTATGGGCACCATTCAGCATCTCAAGTCCAAATTTGGAGATGGCTATA
TCGTCACAATGAAGATCAAATCCCCGAAGGACGACCTGCTTCCTGACCTGAACCCTGTGGAGCAGTTCTTCCAGG
GGAACTTCCCAGGCAGTGTGCAGAGGGAGAGGCACTACAACATGCTCCAGTTCCAGGTCTCCTCCTCCTCCCTGG
CGAGGATCTTCCAGCTCCTCCTCTCCCACAAGGACAGCCTGCTCATCGAGGAGTACTCAGTCACACAGACCACAC
TGGACCAGGTGTTTGTAAATTTTGCTAAACAGCAGACTGAAAGTCATGACCTCCCTCTGCACCCTCGAGCTGCTG
GAGCCAGTCGACAAGCCCAGGACTGATCTTTCACACCGCTCGTTCCTGCAGCCAGAAAGGAACTCTGGGCAGCTG
GAGGCGCAGGAGCCTGTGCCCATATGGTCATCCAAATGGACTGGCCAGCGTAAATGACCCCACTGCAGCAGAAAA
CAAACACACGAGGAGCATGCAGCGAATTCAGAAAGAGGTCTTTCAGAAGGAAACCGAAACTGACTTGCTCACCTG
GAACACCTGATGGTGAAACCAAACAAATACAAAATCCTTCTCCAGACCCCAGAACTAGAAACCCCGGGCCATCCC
ACTAGCAGCTTTGGCCTCCATATTGCTCTCATTTCAAGCAGATCTGCTTTTCTGCATGTTTGTCTGTGTGTCTGC
GTTGTGTGTGATTTTCATGGAAAAATAAAATGCAAATGCACTCATCACAAA
SEQ ID NO: 2
AGGACACAGCGTCCGGAGCCAGAGGCGCTCTTAACGGCGTTTATGTCCTTTGCTGTCTGAGGGGCCTCAGCTCTG
ACCAATCTGGTCTTCGTGTGGTCATTAGCATGGGCTTCGTGAGACAGATACAGCTTTTGCTCTGGAAGAACTGGA
CCCTGCGGAAAAGGCAAAAGATTCGCTTTGTGGTGGAACTCGTGTGGCCTTTATCTTTATTTCTGGTCTTGATCT
GGTTAAGGAATGCCAACCCGCTCTACAGCCATCATGAATGCCATTTCCCCAACAAGGCGATGCCCTCAGCAGGAA
TGCTGCCGTGGCTCCAGGGGATCTTCTGCAATGTGAACAATCCCTGTTTTCAAAGCCCCACCCCAGGAGAATCTC
CTGGAATTGTGTCAAACTATAACAACTCCATCTTGGCAAGGGTATATCGAGATTTTCAAGAACTCCTCATGAATG
CACCAGAGAGCCAGCACCTTGGCCGTATTTGGACAGAGCTACACATCTTGTCCCAATTCATGGACACCCTCCGGA
CTCACCCGGAGAGAATTGCAGGAAGAGGAATACGAATAAGGGATATCTTGAAAGATGAAGAAACACTGACACTAT
TTCTCATTAAAAACATCGGCCTGTCTGACTCAGTGGTCTACCTTCTGATCAACTCTCAAGTCCGTCCAGAGCAGT
TCGCTCATGGAGTCCCGGACCTGGCGCTGAAGGACATCGCCTGCAGCGAGGCCCTCCTGGAGCGCTTCATCATCT
TCAGCCAGAGACGCGGGGCAAAGACGGTGCGCTATGCCCTGTGCTCCCTCTCCCAGGGCACCCTACAGTGGATAG
AAGACACTCTGTATGCCAACGTGGACTTCTTCAAGCTCTTCCGTGTGCTTCCCACACTCCTAGACAGCCGTTCTC
AAGGTATCAATCTGAGATCTTGGGGAGGAATATTATCTGATATGTCACCAAGAATTCAAGAGTTTATCCATCGGC
CGAGTATGCAGGACTTGCTGTGGGTGACCAGGCCCCTCATGCAGAATGGTGGTCCAGAGACCTTTACAAAGCTGA
TGGGCATCCTGTCTGACCTCCTGTGTGGCTACCCCGAGGGAGGTGGCTCTCGGGTGCTCTCCTTCAACTGGTATG
AAGACAATAACTATAAGGCCTTTCTGGGGATTGACTCCACAAGGAAGGATCCTATCTATTCTTATGACAGAAGAA
CAACATCCTTTTGTAATGCATTGATCCAGAGCCTGGAGTCAAATCCTTTAACCAAAATCGCTTGGAGGGCGGCAA
AGCCTTTGCTGATGGGAAAAATCCTGTACACTCCTGATTCACCTGCAGCACGAAGGATACTGAAGAATGCCAACT
CAACTTTTGAAGAACTGGAACACGTTAGGAAGTTGGTCAAAGCCTGGGAAGAAGTAGGGCCCCAGATCTGGTACT
TCTTTGACAACAGCACACAGATGAACATGATCAGAGATACCCTGGGGAACCCAACAGTAAAAGACTTTTTGAATA
GGCAGCTTGGTGAAGAAGGTATTACTGCTGAAGCCATCCTAAACTTCCTCTACAAGGGCCCTCGGGAAAGCCAGG
CTGACGACATGGCCAACTTCGACTGGAGGGACATATTTAACATCACTGATCGCACCCTCCGCCTTGTCAATCAAT
ACCTGGAGTGCTTGGTCCTGGATAAGTTTGAAAGCTACAATGATGAAACTCAGCTCACCCAACGTGCCCTCTCTC
TACTGGAGGAAAACATGTTCTGGGCCGGAGTGGTATTCCCTGACATGTATCCCTGGACCAGCTCTCTACCACCCC
ACGTGAAGTATAAGATCCGAATGGACATAGACGTGGTGGAGAAAACCAATAAGATTAAAGACAGGTATTGGGATT
CTGGTCCCAGAGCTGATCCCGTGGAAGATTTCCGGTACATCTGGGGCGGGTTTGCCTATCTGCAGGACATGGTTG
AACAGGGGATCACAAGGAGCCAGGTGCAGGCGGAGGCTCCAGTTGGAATCTACCTCCAGCAGATGCCCTACCCCT
GCTTCGTGGACGATTCTTTCATGATCATCCTGAACCGCTGTTTCCCTATCTTCATGGTGCTGGCATGGATCTACT
CTGTCTCCATGACTGTGAAGAGCATCGTCTTGGAGAAGGAGTTGCGACTGAAGGAGACCTTGAAAAATCAGGGTG
TCTCCAATGCAGTGATTTGGTGTACCTGGTTCCTGGACAGCTTCTCCATCATGTCGATGAGCATCTTCCTCCTGA
CGATATTCATCATGCATGGAAGAATCCTACATTACAGCGACCCATTCATCCTCTTCCTGTTCTTGTTGGCTTTCT
CCACTGCCACCATCATGCTGTGCTTTCTGCTCAGCACCTTCTTCTCCAAGGCCAGTCTGGCAGCAGCCTGTAGTG
GTGTCATCTATTTCACCCTCTACCTGCCACACATCCTGTGCTTCGCCTGGCAGGACCGCATGACCGCTGAGCTGA
AGAAGGCTGTGAGCTTACTGTCTCCGGTGGCATTTGGATTTGGCACTGAGTACCTGGTTCGCTTTGAAGAGCAAG
GCCTGGGGCTGCAGTGGAGCAACATCGGGAACAGTCCCACGGAAGGGGACGAATTCAGCTTCCTGCTGTCCATGC
AGATGATGCTCCTTGATGCTGCTGTCTATGGCTTACTCGCTTGGTACCTTGATCAGGTGTTTCCAGGAGACTATG
GAACCCCACTTCCTTGGTACTTTCTTCTACAAGAGTCGTATTGGCTTGGCGGTGAAGGGTGTTCAACCAGAGAAG
AAAGAGCCCTGGAAAAGACCGAGCCCCTAACAGAGGAAACGGAGGATCCAGAGCACCCAGAAGGAATACACGACT
CCTTCTTTGAACGTGAGCATCCAGGGTGGGTTCCTGGGGTATGCGTGAAGAATCTGGTAAAGATTTTTGAGCCCT
GTGGCCGGCCAGCTGTGGACCGTCTGAACATCACCTTCTACGAGAACCAGATCACCGCATTCCTGGGCCACAATG
GAGCTGGGAAAACCACCACCTTGTCCATCCTGACGGGTCTGTTGCCACCAACCTCTGGGACTGTGCTCGTTGGGG
GAAGGGACATTGAAACCAGCCTGGATGCAGTCCGGCAGAGCCTTGGCATGTGTCCACAGCACAACATCCTGTTCC
ACCACCTCACGGTGGCTGAGCACATGCTGTTCTATGCCCAGCTGAAAGGAAAGTCCCAGGAGGAGGCCCAGCTGG
AGATGGAAGCCATGTTGGAGGACACAGGCCTCCACCACAAGCGGAATGAAGAGGCTCAGGACCTATCAGGTGGCA
TGCAGAGAAAGCTGTCGGTTGCCATTGCCTTTGTGGGAGATGCCAAGGTGGTGATTCTGGACGAACCCACCTCTG
GGGTGGACCCTTACTCGAGACGCTCAATCTGGGATCTGCTCCTGAAGTATCGCTCAGGCAGAACCATCATCATGT
CCACTCACCACATGGACGAGGCCGACCTCCTTGGGGACCGCATTGCCATCATTGCCCAGGGAAGGCTCTACTGCT
CAGGCACCCCACTCTTCCTGAAGAACTGCTTTGGCACAGGCTTGTACTTAACCTTGGTGCGCAAGATGAAAAACA
TCCAGAGCCAAAGGAAAGGCAGTGAGGGGACCTGCAGCTGCTCGTCTAAGGGTTTCTCCACCACGTGTCCAGCCC
ACGTCGATGACCTAACTCCAGAACAAGTCCTGGATGGGGATGTAAATGAGCTGATGGATGTAGTTCTCCACCATG
TTCCAGAGGCAAAGCTGGTGGAGTGCATTGGTCAAGAACTTATCTTCCTTCTTCCAAATAAGAACTTCAAGCACA
GAGCATATGCCAGCCTTTTCAGAGAGCTGGAGGAGACGCTGGCTGACCTTGGTCTCAGCAGTTTTGGAATTTCTG
ACACTCCCCTGGAAGAGATTTTTCTGAAGGTCACGGAGGATTCTGATTCAGGACCTCTGTTTGCGGGTGGCGCTC
AGCAGAAAAGAGAAAACGTCAACCCCCGACACCCCTGCTTGGGTCCCAGAGAGAAGGCTGGACAGACACCCCAGG
ACTCCAATGTCTGCTCCCCAGGGGCGCCGGCTGCTCACCCAGAGGGCCAGCCTCCCCCAGAGCCAGAGTGCCCAG
GCCCGCAGCTCAACACGGGGACACAGCTGGTCCTCCAGCATGTGCAGGCGCTGCTGGTCAAGAGATTCCAACACA
CCATCCGCAGCCACAAGGACTTCCTGGCGCAGATCGTGCTCCCGGCTACCTTTGTGTTTTTGGCTCTGATGCTTT
CTATTGTTATCCCTCCTTTTGGCGAATACCCCGCTTTGACCCTTCACCCCTGGATATATGGGCAGCAGTACACCT
TCTTCAGCATGGATGAACCAGGCAGTGAGCAGTTCACGGTACTTGCAGACGTCCTCCTGAATAAGCCAGGCTTTG
GCAACCGCTGCCTGAAGGAAGGGTGGCTTCCGGAGTACCCCTGTGGCAACTCAACACCCTGGAAGACTCCTTCTG
TGTCCCCAAACATCACCCAGCTGTTCCAGAAGCAGAAATGGACACAGGTCAACCCTTCACCATCCTGCAGGTGCA
GCACCAGGGAGAAGCTCACCATGCTGCCAGAGTGCCCCGAGGGTGCCGGGGGCCTCCCGCCCCCCCAGAGAACAC
AGCGCAGCACGGAAATTCTACAAGACCTGACGGACAGGAACATCTCCGACTTCTTGGTAAAAACGTATCCTGCTC
TTATAAGAAGCAGCTTAAAGAGCAAATTCTGGGTCAATGAACAGAGGTATGGAGGAATTTCCATTGGAGGAAAGC
TCCCAGTCGTCCCCATCACGGGGGAAGCACTTGTTGGGTTTTTAAGCGACCTTGGCCGGATCATGAATGTGAGCG
GGGGCCCTATCACTAGAGAGGCCTCTAAAGAAATACCTGATTTCCTTAAACATCTAGAAACTGAAGACAACATTA
AGGTGTGGTTTAATAACAAAGGCTGGCATGCCCTGGTCAGCTTTCTCAATGTGGCCCACAACGCCATCTTACGGG
CCAGCCTGCCTAAGGACAGGAGCCCCGAGGAGTATGGAATCACCGTCATTAGCCAACCCCTGAACCTGACCAAGG
AGCAGCTCTCAGAGATTACAGTGCTGACCACTTCAGTGGATGCTGTGGTTGCCATCTGCGTGATTTTCTCCATGT
CCTTCGTCCCAGCCAGCTTTGTCCTTTATTTGATCCAGGAGCGGGTGAACAAATCCAAGCACCTCCAGTTTATCA
GTGGAGTGAGCCCCACCACCTACTGGGTAACCAACTTCCTCTGGGACATCATGAATTATTCCGTGAGTGCTGGGC
TGGTGGTGGGCATCTTCATCGGGTTTCAGAAGAAAGCCTACACTTCTCCAGAAAACCTTCCTGCCCTTGTGGCAC
TGCTCCTGCTGTATGGATGGGCGGTCATTCCCATGATGTACCCAGCATCCTTCCTGTTTGATGTCCCCAGCACAG
CCTATGTGGCTTTATCTTGTGCTAATCTGTTCATCGGCATCAACAGCAGTGCTATTACCTTCATCTTGGAATTAT
TTGAGAATAACCGGACGCTGCTCAGGTTCAACGCCGTGCTGAGGAAGCTGCTCATTGTCTTCCCCCACTTCTGCC
TGGGCCGGGGCCTCATTGACCTTGCACTGAGCCAGGCTGTGACAGATGTCTATGCCCGGTTTGGTGAGGAGCACT
CTGCAAATCCGTTCCACTGGGACCTGATTGGGAAGAACCTGTTTGCCATGGTGGTGGAAGGGGTGGTGTACTTCC
TCCTGACCCTGCTGGTCCAGCGCCACTTCTTCCTCTCCCAATGGATTGCCGAGCCCACTAAGGAGCCCATTGTTG
ATGAAGATGATGATGTGGCTGAAGAAAGACAAAGAATTATTACTGGTGGAAATAAAACTGACATCTTAAGGCTAC
ATGAACTAACCAAGATTTATCCAGGCACCTCCAGCCCAGCAGTGGACAGGCTGTGTGTCGGAGTTCGCCCTGGAG
AGTGCTTTGGCCTCCTGGGAGTGAATGGTGCCGGCAAAACAACCACATTCAAGATGCTCACTGGGGACACCACAG
TGACCTCAGGGGATGCCACCGTAGCAGGCAAGAGTATTTTAACCAATATTTCTGAAGTCCATCAAAATATGGGCT
ACTGTCCTCAGTTTGATGCAATCGATGAGCTGCTCACAGGACGAGAACATCTTTACCTTTATGCCCGGCTTCGAG
GTGTACCAGCAGAAGAAATCGAAAAGGTTGCAAACTGGAGTATTAAGAGCCTGGGCCTGACTGTCTACGCCGACT
GCCTGGCTGGCACGTACAGTGGGGGCAACAAGCGGAAACTCTCCACAGCCATCGCACTCATTGGCTGCCCACCGC
TGGTGCTGCTGGATGAGCCCACCACAGGGATGGACCCCCAGGCACGCCGCATGCTGTGGAACGTCATCGTGAGCA
TCATCAGAGAAGGGAGGGCTGTGGTCCTCACATCCCACAGCATGGAAGAATGTGAGGCACTGTGTACCCGGCTGG
CCATCATGGTAAAGGGCGCCTTTCGATGTATGGGCACCATTCAGCATCTCAAGTCCAAATTTGGAGATGGCTATA
TCGTCACAATGAAGATCAAATCCCCGAAGGACGACCTGCTTCCTGACCTGAACCCTGTGGAGCAGTTCTTCCAGG
GGAACTTCCCAGGCAGTGTGCAGAGGGAGAGGCACTACAACATGCTCCAGTTCCAGGTCTCCTCCTCCTCCCTGG
CGAGGATCTTCCAGCTCCTCCTCTCCCACAAGGACAGCCTGCTCATCGAGGAGTACTCAGTCACACAGACCACAC
TGGACCAGGTGTTTGTAAATTTTGCTAAACAGCAGACTGAAAGTCATGACCTCCCTCTGCACCCTCGAGCTGCTG
GAGCCAGTCGACAAGCCCAGGACTGATCTTTCACACCGCTCGTTCCTGCAGCCAGAAAGGAACTCTGGGCAGCTG
GAGGCGCAGGAGCCTGTGCCCATATGGTCATCCAAATGGACTGGCCAGCGTAAATGACCCCACTGCAGCAGAAAA
CAAACACACGAGGAGCATGCAGCGAATTCAGAAAGAGGTCTTTCAGAAGGAAACCGAAACTGACTTGCTCACCTG
GAACACCTGATGGTGAAACCAAACAAATACAAAATCCTTCTCCAGACCCCAGAACTAGAAACCCCGGGCCATCCC
ACTAGCAGCTTTGGCCTCCATATTGCTCTCATTTCAAGCAGATCTGCTTTTCTGCATGTTTGTCTGTGTGTCTGC
GTTGTGTGTGATTTTCATGGAAAAATAAAATGCAAATGCACTCATCACAAA
SEQ ID NO: 3
TTGGCCACTCCCTCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAGAGAGGGAGTGGCCAACTCCATCACTAGGGGTTCCTGCGGC
AATTCAGTCGATAACTATAACGGTCCTAAGGTAGCGATTTAAATGGTACCGGGCCCCAGAAGCCTGGTGGTTGTT
TGTCCTTCTCAGGGGAAAAGTGAGGCGGCCCCTTGGAGGAAGGGGCCGGGCAGAATGATCTAATCGGATTCCAAG
CAGCTCAGGGGATTGTCTTTTTCTAGCACCTTCTTGCCACTCCTAAGCGTCCTCCGTGACCCCGGCTGGGATTTA
GCCTGGTGCTGTGTCAGCCCCGGGTGCCGCAGGGGGACGGCTGCCTTCGGGGGGGACGGGGCAGGGCGGGGTTCG
GCTTCTGGCGTGTGACCGGCGGCTCTAGAGCCTCTGCTAACCATGTTCATGCCTTCTTCTTTTTCCTACAGCTCC
TGGGCAACGTGCTGGTTATTGTGCTGTCTCATCATTTTGGCAAAGAATTACCACCATGGGCTTCGTGAGACAGAT
ACAGCTTTTGCTCTGGAAGAACTGGACCCTGCGGAAAAGGCAAAAGATTCGCTTTGTGGTGGAACTCGTGTGGCC
TTTATCTTTATTTCTGGTCTTGATCTGGTTAAGGAATGCCAACCCGCTCTACAGCCATCATGAATGCCATTTCCC
CAACAAGGCGATGCCCTCAGCAGGAATGCTGCCGTGGCTCCAGGGGATCTTCTGCAATGTGAACAATCCCTGTTT
TCAAAGCCCCACCCCAGGAGAATCTCCTGGAATTGTGTCAAACTATAACAACTCCATCTTGGCAAGGGTATATCG
AGATTTTCAAGAACTCCTCATGAATGCACCAGAGAGCCAGCACCTTGGCCGTATTTGGACAGAGCTACACATCTT
GTCCCAATTCATGGACACCCTCCGGACTCACCCGGAGAGAATTGCAGGAAGAGGAATACGAATAAGGGATATCTT
GAAAGATGAAGAAACACTGACACTATTTCTCATTAAAAACATCGGCCTGTCTGACTCAGTGGTCTACCTTCTGAT
CAACTCTCAAGTCCGTCCAGAGCAGTTCGCTCATGGAGTCCCGGACCTGGCGCTGAAGGACATCGCCTGCAGCGA
GGCCCTCCTGGAGCGCTTCATCATCTTCAGCCAGAGACGCGGGGCAAAGACGGTGCGCTATGCCCTGTGCTCCCT
CTCCCAGGGCACCCTACAGTGGATAGAAGACACTCTGTATGCCAACGTGGACTTCTTCAAGCTCTTCCGTGTGCT
TCCCACACTCCTAGACAGCCGTTCTCAAGGTATCAATCTGAGATCTTGGGGAGGAATATTATCTGATATGTCACC
AAGAATTCAAGAGTTTATCCATCGGCCGAGTATGCAGGACTTGCTGTGGGTGACCAGGCCCCTCATGCAGAATGG
TGGTCCAGAGACCTTTACAAAGCTGATGGGCATCCTGTCTGACCTCCTGTGTGGCTACCCCGAGGGAGGTGGCTC
TCGGGTGCTCTCCTTCAACTGGTATGAAGACAATAACTATAAGGCCTTTCTGGGGATTGACTCCACAAGGAAGGA
TCCTATCTATTCTTATGACAGAAGAACAACATCCTTTTGTAATGCATTGATCCAGAGCCTGGAGTCAAATCCTTT
AACCAAAATCGCTTGGAGGGCGGCAAAGCCTTTGCTGATGGGAAAAATCCTGTACACTCCTGATTCACCTGCAGC
ACGAAGGATACTGAAGAATGCCAACTCAACTTTTGAAGAACTGGAACACGTTAGGAAGTTGGTCAAAGCCTGGGA
AGAAGTAGGGCCCCAGATCTGGTACTTCTTTGACAACAGCACACAGATGAACATGATCAGAGATACCCTGGGGAA
CCCAACAGTAAAAGACTTTTTGAATAGGCAGCTTGGTGAAGAAGGTATTACTGCTGAAGCCATCCTAAACTTCCT
CTACAAGGGCCCTCGGGAAAGCCAGGCTGACGACATGGCCAACTTCGACTGGAGGGACATATTTAACATCACTGA
TCGCACCCTCCGCCTTGTCAATCAATACCTGGAGTGCTTGGTCCTGGATAAGTTTGAAAGCTACAATGATGAAAC
TCAGCTCACCCAACGTGCCCTCTCTCTACTGGAGGAAAACATGTTCTGGGCCGGAGTGGTATTCCCTGACATGTA
TCCCTGGACCAGCTCTCTACCACCCCACGTGAAGTATAAGATCCGAATGGACATAGACGTGGTGGAGAAAACCAA
TAAGATTAAAGACAGGTATTGGGATTCTGGTCCCAGAGCTGATCCCGTGGAAGATTTCCGGTACATCTGGGGCGG
GTTTGCCTATCTGCAGGACATGGTTGAACAGGGGATCACAAGGAGCCAGGTGCAGGCGGAGGCTCCAGTTGGAAT
CTACCTCCAGCAGATGCCCTACCCCTGCTTCGTGGACGATTCTTTCATGATCATCCTGAACCGCTGTTTCCCTAT
CTTCATGGTGCTGGCATGGATCTACTCTGTCTCCATGACTGTGAAGAGCATCGTCTTGGAGAAGGAGTTGCGACT
GAAGGAGACCTTGAAAAATCAGGGTGTCTCCAATGCAGTGATTTGGTGTACCTGGTTCCTGGACAGCTTCTCCAT
CATGTCGATGAGCATCTTCCTCCTGACGATATTCATCATGCATGGAAGAATCCTACATTACAGCGACCCATTCAT
CCTCTTCCTGTTCTTGTTGGCTTTCTCCACTGCCACCATCATGCTGTGCTTTCTGCTCAGCACCTTCTTCTCCAA
GGCCAGTCTGGCAGCAGCCTGTAGTGGTGTCATCTATTTCACCCTCTACCTGCCACACATCCTGTGCTTCGCCTG
GCAGGACCGCATGACCGCTGAGCTGAAGAAGGCTGTGAGCTTACTGTCTCCGGTGGCATTTGGATTTGGCACTGA
GTACCTGGTTCGCTTTGAAGAGCAAGGCCTGGGGCTGCAGTGGAGCAACATCGGGAACAGTCCCACGGAAGGGGA
CGAATTCAGCTTCCTGCTGTCCATGCAGATGATGCTCCTTGATGCTGCTGTCTATGGCTTACTCGCTTGGTACCT
TGATCAGGTGTTTCCAGGAGACTATGGAACCCCACTTCCTTGGTACTTTCTTCTACAAGAGTCGTATTGGCTTGG
CGGTGAAGGGTGTTCAACCAGAGAAGAAAGAGCCCTGGAAAAGACCGAGCCCCTAACAGAGGAAACGGAGGATCC
AGAGCACCCAGAAGGAATACACGACTCCTTCTTTGAACGTGAGCATCCAGGGTGGGTTCCTGGGGTATGCGTGAA
GAATCTGGTAAAGATTTTTGAGCCCTGTGGCCGGCCAGCTGTGGACCGTCTGAACATCACCTTCTACGAGAACCA
GATCACCGCATTCCTGGGCCACAATGGAGCTGGGAAAACCACCACCTTGTCCATCCTGACGGGTCTGTTGCCACC
AACCTCTGGGACTGTGCTCGTTGGGGGAAGGGACATTGAAACCAGCCTGGATGCAGTCCGGCAGAGCCTTGGCAT
GTGTCCACAGCACAACATCCTGTTCCACCACCTCACGGTGGCTGAGCACATGCTGTTCTATGCCCAGCTGAAAGG
AAAGTCCCAGGAGGAGGCCCAGCTGGAGATGGAAGCCATGTTGGAGGACACAGGCCTCCACCACAAGCGGAATGA
AGAGGCTCAGGACCTATCAGGTGGCATGCAGAGAAAGCTGTCGGTTGCCATTGCCTTTGTGGGAGATGCCAAGGT
GGTGATTCTGGACGAACCCACCTCTGGGGTGGACCCTTACTCGAGACGCTCAATCTGGAATCTGCTCCTGAAGTA
TCGCTCAGGCAGAACCATCATCATGTCCACTCACCACATGGACGAGGCCGACCTCCTTGGGGACCGCATTGCCAT
CATTGCCCAGGGAAGGCTCTACTGCTCAGGCACCCCACTCTTCCTGAAGAACTGCTTTGGCACAGGCTTGTACTT
AACCTTGGTGCGCAAGATGAAAAACATCCAGAGCCAAAGGAAAGGCAGTGAGGGGACCTGCAGCTGCTCGTCTAA
GGGTTTCTCCACCACGTGTCCAGCCCACGTCGATGACCTAACTCCAGAACAAGTCCTGGATGGGGATGTAAATGA
GCTGATGGATGTAGTTCTCCACCATGTTCCAGAGGCAAAGCTGGTGGAGTGCATTGGTCAAGAACTTATCTTCCT
TCTTCCATTTAAATTAGGGATAACAGGGTAATGGCGCGGGCCGCAGGAACCCCTAGTGATGGAGTTGGCCACTCC
CTCTCTGCGCGCTCGCTCGCTCACTGAGGCCGCCCGGGCAAAGCCCGGGCGTCGGGCGACCTTTGGTCGCCCGGC
CTCAGTGAGCGAGCGAGCGCGCAGAGAGGGAGTGGCCAA
SEQ ID NO: 4
TTGGCCACTCCCTCTCTGCGCGCTCGCTCGCTCACTGAGGCCGGGCGACCAAAGGTCGCCCGACGCCCGGGCTTT
GCCCGGGCGGCCTCAGTGAGCGAGCGAGCGCGCAGAGAGGGAGTGGCCAACTCCATCACTAGGGGTTCCTGCGGC
AATTCAGTCGATAACTATAACGGTCCTAAGGTAGCGATTTAAATAACATCCAGAGCCAAAGGAAAGGCAGTGAGG
GGACCTGCAGCTGCTCGTCTAAGGGTTTCTCCACCACGTGTCCAGCCCACGTCGATGACCTAACTCCAGAACAAG
TCCTGGATGGGGATGTAAATGAGCTGATGGATGTAGTTCTCCACCATGTTCCAGAGGCAAAGCTGGTGGAGTGCA
TTGGTCAAGAACTTATCTTCCTTCTTCCAAATAAGAACTTCAAGCACAGAGCATATGCCAGCCTTTTCAGAGAGC
TGGAGGAGACGCTGGCTGACCTTGGTCTCAGCAGTTTTGGAATTTCTGACACTCCCCTGGAAGAGATTTTTCTGA
AGGTCACGGAGGATTCTGATTCAGGACCTCTGTTTGCGGGTGGCGCTCAGCAGAAAAGAGAAAACGTCAACCCCC
GACACCCCTGCTTGGGTCCCAGAGAGAAGGCTGGACAGACACCCCAGGACTCCAATGTCTGCTCCCCAGGGGCGC
CGGCTGCTCACCCAGAGGGCCAGCCTCCCCCAGAGCCAGAGTGCCCAGGCCCGCAGCTCAACACGGGGACACAGC
TGGTCCTCCAGCATGTGCAGGCGCTGCTGGTCAAGAGATTCCAACACACCATCCGCAGCCACAAGGACTTCCTGG
CGCAGATCGTGCTCCCGGCTACCTTTGTGTTTTTGGCTCTGATGCTTTCTATTGTTATCCCTCCTTTTGGCGAAT
ACCCCGCTTTGACCCTTCACCCCTGGATATATGGGCAGCAGTACACCTTCTTCAGCATGGATGAACCAGGCAGTG
AGCAGTTCACGGTACTTGCAGACGTCCTCCTGAATAAGCCAGGCTTTGGCAACCGCTGCCTGAAGGAAGGGTGGC
TTCCGGAGTACCCCTGTGGCAACTCAACACCCTGGAAGACTCCTTCTGTGTCCCCAAACATCACCCAGCTGTTCC
AGAAGCAGAAATGGACACAGGTCAACCCTTCACCATCCTGCAGGTGCAGCACCAGGGAGAAGCTCACCATGCTGC
CAGAGTGCCCCGAGGGTGCCGGGGGCCTCCCGCCCCCCCAGAGAACACAGCGCAGCACGGAAATTCTACAAGACC
TGACGGACAGGAACATCTCCGACTTCTTGGTAAAAACGTATCCTGCTCTTATAAGAAGCAGCTTAAAGAGCAAAT
TCTGGGTCAATGAACAGAGGTATGGAGGAATTTCCATTGGAGGAAAGCTCCCAGTCGTCCCCATCACGGGGGAAG
CACTTGTTGGGTTTTTAAGCGACCTTGGCCGGATCATGAATGTGAGCGGGGGCCCTATCACTAGAGAGGCCTCTA
AAGAAATACCTGATTTCCTTAAACATCTAGAAACTGAAGACAACATTAAGGTGTGGTTTAATAACAAAGGCTGGC
ATGCCCTGGTCAGCTTTCTCAATGTGGCCCACAACGCCATCTTACGGGCCAGCCTGCCTAAGGACAGGAGCCCCG
AGGAGTATGGAATCACCGTCATTAGCCAACCCCTGAACCTGACCAAGGAGCAGCTCTCAGAGATTACAGTGCTGA
CCACTTCAGTGGATGCTGTGGTTGCCATCTGCGTGATTTTCTCCATGTCCTTCGTCCCAGCCAGCTTTGTCCTTT
ATTTGATCCAGGAGCGGGTGAACAAATCCAAGCACCTCCAGTTTATCAGTGGAGTGAGCCCCACCACCTACTGGG
TAACCAACTTCCTCTGGGACATCATGAATTATTCCGTGAGTGCTGGGCTGGTGGTGGGCATCTTCATCGGGTTTC
AGAAGAAAGCCTACACTTCTCCAGAAAACCTTCCTGCCCTTGTGGCACTGCTCCTGCTGTATGGATGGGCGGTCA
TTCCCATGATGTACCCAGCATCCTTCCTGTTTGATGTCCCCAGCACAGCCTATGTGGCTTTATCTTGTGCTAATC
TGTTCATCGGCATCAACAGCAGTGCTATTACCTTCATCTTGGAATTATTTGAGAATAACCGGACGCTGCTCAGGT
TCAACGCCGTGCTGAGGAAGCTGCTCATTGTCTTCCCCCACTTCTGCCTGGGCCGGGGCCTCATTGACCTTGCAC
TGAGCCAGGCTGTGACAGATGTCTATGCCCGGTTTGGTGAGGAGCACTCTGCAAATCCGTTCCACTGGGACCTGA
TTGGGAAGAACCTGTTTGCCATGGTGGTGGAAGGGGTGGTGTACTTCCTCCTGACCCTGCTGGTCCAGCGCCACT
TCTTCCTCTCCCAATGGATTGCCGAGCCCACTAAGGAGCCCATTGTTGATGAAGATGATGATGTGGCTGAAGAAA
GACAAAGAATTATTACTGGTGGAAATAAAACTGACATCTTAAGGCTACATGAACTAACCAAGATTTATCCAGGCA
CCTCCAGCCCAGCAGTGGACAGGCTGTGTGTCGGAGTTCGCCCTGGAGAGTGCTTTGGCCTCCTGGGAGTGAATG
GTGCCGGCAAAACAACCACATTCAAGATGCTCACTGGGGACACCACAGTGACCTCAGGGGATGCCACCGTAGCAG
GCAAGAGTATTTTAACCAATATTTCTGAAGTCCATCAAAATATGGGCTACTGTCCTCAGTTTGATGCAATCGATG
AGCTGCTCACAGGACGAGAACATCTTTACCTTTATGCCCGGCTTCGAGGTGTACCAGCAGAAGAAATCGAAAAGG
TTGCAAACTGGAGTATTAAGAGCCTGGGCCTGACTGTCTACGCCGACTGCCTGGCTGGCACGTACAGTGGGGGCA
ACAAGCGGAAACTCTCCACAGCCATCGCACTCATTGGCTGCCCACCGCTGGTGCTGCTGGATGAGCCCACCACAG
GGATGGACCCCCAGGCACGCCGCATGCTGTGGAACGTCATCGTGAGCATCATCAGAGAAGGGAGGGCTGTGGTCC
TCACATCCCACAGCATGGAAGAATGTGAGGCACTGTGTACCCGGCTGGCCATCATGGTAAAGGGCGCCTTTCGAT
GTATGGGCACCATTCAGCATCTCAAGTCCAAATTTGGAGATGGCTATATCGTCACAATGAAGATCAAATCCCCGA
AGGACGACCTGCTTCCTGACCTGAACCCTGTGGAGCAGTTCTTCCAGGGGAACTTCCCAGGCAGTGTGCAGAGGG
AGAGGCACTACAACATGCTCCAGTTCCAGGTCTCCTCCTCCTCCCTGGCGAGGATCTTCCAGCTCCTCCTCTCCC
ACAAGGACAGCCTGCTCATCGAGGAGTACTCAGTCACACAGACCACACTGGACCAGGTGTTTGTAAATTTTGCTA
AACAGCAGACTGAAAGTCATGACCTCCCTCTGCACCCTCGAGCTGCTGGAGCCAGTCGACAAGCCCAGGACTGAA
AGCTTATCGATAATCAACCTCTGGATTACAAAATTTGTGAAAGATTGACTGGTATTCTTAACTATGTTGCTCCTT
TTACGCTATGTGGATACGCTGCTTTAATGCCTTTGTATCATGCTATTGCTTCCCGTATGGCTTTCATTTTCTCCT
CCTTGTATAAATCCTGGTTGCTGTCTCTTTATGAGGAGTTGTGGCCCGTTGTCAGGCAACGTGGCGTGGTGTGCA
CTGTGTTTGCTGACGCAACCCCCACTGGTTGGGGCATTGCCACCACCTGTCAGCTCCTTTCCGGGACTTTCGCTT
TCCCCCTCCCTATTGCCACGGCGGAACTCATCGCCGCCTGCCTTGCCCGCTGCTGGACAGGGGCTCGGCTGTTGG
GCACTGACAATTCCGTGGTGTTGTCGGGGAAATCATCGTCCTTTCCTTGGCTGCTCGCCTGTGTTGCCACCTGGA
TTCTGCGCGGGACGTCCTTCTGCTACGTCCCTTCGGCCCTCAATCCAGCGGACCTTCCTTCCCGCGGCCTGCTGC
CGGCTCTGCGGCCTCTTCCGCGTCTTCGCCTTCGCCCTCAGACGAGTCGGATCTCCCTTTGGGCCGCCTCCCCGC
ATGCCGCTGATCAGCCTCGACTGTGCCTTCTAGTTGCCAGCCATCTGTTGTTTGCCCCTCCCCCGTGCCTTCCTT
GACCCTGGAAGGTGCCACTCCCACTGTCCTTTCCTAATAAAATGAGGAAATTGCATCGCATTGTCTGAGTAGGTG
TCATTCTATTCTGGGGGGTGGGGTGGGGCAGGACAGCAAGGGGGAGGATTGGGAAGACAATAGCAGGCATGCTGG
GGATGCGGTGGGCTCTATGGCTTCTGAGGCGGAAAGAACCAGCTGGGGATTTAAATTAGGGATAACAGGGTAATG
GCGCGGGCCGCAGGAACCCCTAGTGATGGAGTTGGCCACTCCCTCTCTGCGCGCTCGCTCGCTCACTGAGGCCGC
CCGGGCAAAGCCCGGGCGTCGGGCGACCTTTGGTCGCCCGGCCTCAGTGAGCGAGCGAGCGCGCAGAGAGGGAGT
GGCCAA
SEQ ID NO: 5
GGGCCCCAGAAGCCTGGTGGTTGTTTGTCCTTCTCAGGGGAAAAGTGAGGCGGCCCCTTGGAGGAAGGGGCCGGG
CAGAATGATCTAATCGGATTCCAAGCAGCTCAGGGGATTGTCTTTTTCTAGCACCTTCTTGCCACTCCTAAGCGT
CCTCCGTGACCCCGGCTGGGATTTAGCCTGGTGCTGTGTCAGCCCCGGG
SEQ ID NO: 6
GTGCCGCAGGGGGACGGCTGCCTTCGGGGGGGACGGGGCAGGGCGGGGTTCGGCTTCTGGCGTGTGACCGGCGGC
TCTAGAGCCTCTGCTAACCATGTTCATGCCTTCTTCTTTTTCCTACAGCTCCTGGGCAACGTGCTGGTTATTGTG
CTGTCTCATCATTTTGGCAAAGAATTACCACCATGG
SEQ ID NO: 7
ATCGATAATCAACCTCTGGATTACAAAATTTGTGAAAGATTGACTGGTATTCTTAACTATGTTGCTCCTTTTACG
CTATGTGGATACGCTGCTTTAATGCCTTTGTATCATGCTATTGCTTCCCGTATGGCTTTCATTTTCTCCTCCTTG
TATAAATCCTGGTTGCTGTCTCTTTATGAGGAGTTGTGGCCCGTTGTCAGGCAACGTGGCGTGGTGTGCACTGTG
TTTGCTGACGCAACCCCCACTGGTTGGGGCATTGCCACCACCTGTCAGCTCCTTTCCGGGACTTTCGCTTTCCCC
CTCCCTATTGCCACGGCGGAACTCATCGCCGCCTGCCTTGCCCGCTGCTGGACAGGGGCTCGGCTGTTGGGCACT
GACAATTCCGTGGTGTTGTCGGGGAAATCATCGTCCTTTCCTTGGCTGCTCGCCTGTGTTGCCACCTGGATTCTG
CGCGGGACGTCCTTCTGCTACGTCCCTTCGGCCCTCAATCCAGCGGACCTTCCTTCCCGCGGCCTGCTGCCGGCT
CTGCGGCCTCTTCCGCGTCTTCGCCTTCGCCCTCAGACGAGTCGGATCTCCCTTTGGGCCGCCTCCCC
SEQ ID NO: 8
CGCTGATCAGCCTCGACTGTGCCTTCTAGTTGCCAGCCATCTGTTGTTTGCCCCTCCCCCGTGCCTTCCTTGACC
CTGGAAGGTGCCACTCCCACTGTCCTTTCCTAATAAAATGAGGAAATTGCATCGCATTGTCTGAGTAGGTGTCAT
TCTATTCTGGGGGGTGGGGTGGGGCAGGACAGCAAGGGGGAGGATTGGGAAGACAATAGCAGGCATGCTGGGGAT
GCGGTGGGCTCTATGGCTTCTGAGGCGGAAAGAACCAGCTGGGG
SEQ ID NO: 9
GGTACCGGGCCCCAGAAGCCTGGTGGTTGTTTGTCCTTCTCAGGGGAAAAGTGAGGCGGCCCCTTGGAGGAAGGG
GCCGGGCAGAATGATCTAATCGGATTCCAAGCAGCTCAGGGGATTGTCTTTTTCTAGCACCTTCTTGCCACTCCT
AAGCGTCCTCCGTGACCCCGGCTGGGATTTAGCCTGGTGCTGTGTCAGCCCCGGGTGCCGCAGGGGGACGGCTGC
CTTCGGGGGGGACGGGGCAGGGCGGGGTTCGGCTTCTGGCGTGTGACCGGCGGCTCTAGAGCCTCTGCTAACCAT
GTTCATGCCTTCTTCTTTTTCCTACAGCTCCTGGGCAACGTGCTGGTTATTGTGCTGTCTCATCATTTTGGCAAA
GAATTACCACCATGGGCTTCGTGAGACAGATACAGCTTTTGCTCTGGAAGAACTGGACCCTGCGGAAAAGGCAAA
AGATTCGCTTTGTGGTGGAACTCGTGTGGCCTTTATCTTTATTTCTGGTCTTGATCTGGTTAAGGAATGCCAACC
CGCTCTACAGCCATCATGAATGCCATTTCCCCAACAAGGCGATGCCCTCAGCAGGAATGCTGCCGTGGCTCCAGG
GGATCTTCTGCAATGTGAACAATCCCTGTTTTCAAAGCCCCACCCCAGGAGAATCTCCTGGAATTGTGTCAAACT
ATAACAACTCCATCTTGGCAAGGGTATATCGAGATTTTCAAGAACTCCTCATGAATGCACCAGAGAGCCAGCACC
TTGGCCGTATTTGGACAGAGCTACACATCTTGTCCCAATTCATGGACACCCTCCGGACTCACCCGGAGAGAATTG
CAGGAAGAGGAATACGAATAAGGGATATCTTGAAAGATGAAGAAACACTGACACTATTTCTCATTAAAAACATCG
GCCTGTCTGACTCAGTGGTCTACCTTCTGATCAACTCTCAAGTCCGTCCAGAGCAGTTCGCTCATGGAGTCCCGG
ACCTGGCGCTGAAGGACATCGCCTGCAGCGAGGCCCTCCTGGAGCGCTTCATCATCTTCAGCCAGAGACGCGGGG
CAAAGACGGTGCGCTATGCCCTGTGCTCCCTCTCCCAGGGCACCCTACAGTGGATAGAAGACACTCTGTATGCCA
ACGTGGACTTCTTCAAGCTCTTCCGTGTGCTTCCCACACTCCTAGACAGCCGTTCTCAAGGTATCAATCTGAGAT
CTTGGGGAGGAATATTATCTGATATGTCACCAAGAATTCAAGAGTTTATCCATCGGCCGAGTATGCAGGACTTGC
TGTGGGTGACCAGGCCCCTCATGCAGAATGGTGGTCCAGAGACCTTTACAAAGCTGATGGGCATCCTGTCTGACC
TCCTGTGTGGCTACCCCGAGGGAGGTGGCTCTCGGGTGCVTCTCCTTCAACTGGTATGAAGACAATAACTATAAG
CCTTTCTGGGGATTGACTCCACAAGGAAGGATCCTATCTATTCTTATGACAGAAGAACAACATCCTTTTGTAATG
CATTGATCCAGAGCCTGGAGTCAAATCCTTTAACCAAAATCGCTTGGAGGGCGGCAAAGCCTTTGCTGATGGGAA
AAATCCTGTACACTCCTGATTCACCTGCAGCACGAAGGATACTGAAGAATGCCAACTCAACTTTTGAAGAACTGG
AACACGTTAGGAAGTTGGTCAAAGCCTGGGAAGAAGTAGGGCCCCAGATCTGGTACTTCTTTGACAACAGCACAC
AGATGAACATGATCAGAGATACCCTGGGGAACCCAACAGTAAAAGACTTTTTGAATAGGCAGCTTGGTGAAGAAG
GTATTACTGCTGAAGCCATCCTAAACTTCCTCTACAAGGGCCCTCGGGAAAGCCAGGCTGACGACATGGCCAACT
TCGACTGGAGGGACATATTTAACATCACTGATCGCACCCTCCGCCTTGTCAATCAATACCTGGAGTGCTTGGTCC
TGGATAAGTTTGAAAGCTACAATGATGAAACTCAGCTCACCCAACGTGCCCTCTCTCTACTGGAGGAAAACATGT
TCTGGGCCGGAGTGGTATTCCCTGACATGTATCCCTGGACCAGCTCTCTACCACCCCACGTGAAGTATAAGATCC
GAATGGACATAGACGTGGTGGAGAAAACCAATAAGATTAAAGACAGGTATTGGGATTCTGGTCCCAGAGCTGATC
CCGTGGAAGATTTCCGGTACATCTGGGGCGGGTTTGCCTATCTGCAGGACATGGTTGAACAGGGGATCACAAGGA
GCCAGGTGCAGGCGGAGGCTCCAGTTGGAATCTACCTCCAGCAGATGCCCTACCCCTGCTTCGTGGACGATTCTT
TCATGATCATCCTGAACCGCTGTTTCCCTATCTTCATGGTGCTGGCATGGATCTACTCTGTCTCCATGACTGTGA
AGAGCATCGTCTTGGAGAAGGAGTTGCGACTGAAGGAGACCTTGAAAAATCAGGGTGTCTCCAATGCAGTGATTT
GGTGTACCTGGTTCCTGGACAGCTTCTCCATCATGTCGATGAGCATCTTCCTCCTGACGATATTCATCATGCATG
GAAGAATCCTACATTACAGCGACCCATTCATCCTCTTCCTGTTCTTGTTGGCTTTCTCCACTGCCACCATCATGC
TGTGCTTTCTGCTCAGCACCTTCTTCTCCAAGGCCAGTCTGGCAGCAGCCTGTAGTGGTGTCATCTATTTCACCC
TCTACCTGCCACACATCCTGTGCTTCGCCTGGCAGGACCGCATGACCGCTGAGCTGAAGAAGGCTGTGAGCTTAC
TGTCTCCGGTGGCATTTGGATTTGGCACTGAGTACCTGGTTCGCTTTGAAGAGCAAGGCCTGGGGCTGCAGTGGA
GCAACATCGGGAACAGTCCCACGGAAGGGGACGAATTCAGCTTCCTGCTGTCCATGCAGATGATGCTCCTTGATG
CTGCTGTCTATGGCTTACTCGCTTGGTACCTTGATCAGGTGTTTCCAGGAGACTATGGAACCCCACTTCCTTGGT
ACTTTCTTCTACAAGAGTCGTATTGGCTTGGCGGTGAAGGGTGTTCAACCAGAGAAGAAAGAGCCCTGGAAAAGA
CCGAGCCCCTAACAGAGGAAACGGAGGATCCAGAGCACCCAGAAGGAATACACGACTCCTTCTTTGAACGTGAGC
ATCCAGGGTGGGTTCCTGGGGTATGCGTGAAGAATCTGGTAAAGATTTTTGAGCCCTGTGGCCGGCCAGCTGTGG
ACCGTCTGAACATCACCTTCTACGAGAACCAGATCACCGCATTCCTGGGCCACAATGGAGCTGGGAAAACCACCA
CCTTGTCCATCCTGACGGGTCTGTTGCCACCAACCTCTGGGACTGTGCTCGTTGGGGGAAGGGACATTGAAACCA
GCCTGGATGCAGTCCGGCAGAGCCTTGGCATGTGTCCACAGCACAACATCCTGTTCCACCACCTCACGGTGGCTG
AGCACATGCTGTTCTATGCCCAGCTGAAAGGAAAGTCCCAGGAGGAGGCCCAGCTGGAGATGGAAGCCATGTTGG
AGGACACAGGCCTCCACCACAAGCGGAATGAAGAGGCTCAGGACCTATCAGGTGGCATGCAGAGAAAGCTGTCGG
TTGCCATTGCCTTTGTGGGAGATGCCAAGGTGGTGATTCTGGACGAACCCACCTCTGGGGTGGACCCTTACTCGA
GACGCTCAATCTGGGATCTGCTCCTGAAGTATCGCTCAGGCAGAACCATCATCATGTCCACTCACCACATGGACG
AGGCCGACCTCCTTGGGGACCGCATTGCCATCATTGCCCAGGGAAGGCTCTACTGCTCAGGCACCCCACTCTTCC
TGAAGAACTGCTTTGGCACAGGCTTGTACTTAACCTTGGTGCGCAAGATGAAAAACATCCAGAGCCAAAGGAAAG
GCAGTGAGGGGACCTGCAGCTGCTCGTCTAAGGGTTTCTCCACCACGTGTCCAGCCCACGTCGATGACCTAACTC
CAGAACAAGTCCTGGATGGGGATGTAAATGAGCTGATGGATGTAGTTCTCCACCATGTTCCAGAGGCAAAGCTGG
TGGAGTGCATTGGTCAAGAACTTATCTTCCTTCTTCC
SEQ ID NO: 10
ACATCCAGAGCCAAAGGAAAGGCAGTGAGGGGACCTGCAGCTGCTCGTCTAAGGGTTTCTCCACCACGTGT
CCAGCCCACGTCGATGACCTAACTCCAGAACAAGTCCTGGATGGGGATGTAAATGAGCTGATGGATGTAGT
TCTCCACCATGTTCCAGAGGCAAAGCTGGTGGAGTGCATTGGTCAAGAACTTATCTTCCTTCTTCCAAATA
AGAACTTCAAGCACAGAGCATATGCCAGCCTTTTCAGAGAGCTGGAGGAGACGCTGGCTGACCTTGGTCTC
AGCAGTTTTGGAATTTCTGACACTCCCCTGGAAGAGATTTTTCTGAAGGTCACGGAGGATTCTGATTCAGG
ACCTCTGTTTGCGGGTGGCGCTCAGCAGAAAAGAGAAAACGTCAACCCCCGACACCCCTGCTTGGGTCCCA
GAGAGAAGGCTGGACAGACACCCCAGGACTCCAATGTCTGCTCCCCAGGGGCGCCGGCTGCTCACCCAGAG
GGCCAGCCTCCCCCAGAGCCAGAGTGCCCAGGCCCGCAGCTCAACACGGGGACACAGCTGGTCCTCCAGCA
TGTGCAGGCGCTGCTGGTCAAGAGATTCCAACACACCATCCGCAGCCACAAGGACTTCCTGGCGCAGATCG
TGCTCCCGGCTACCTTTGTGTTTTTGGCTCTGATGCTTTCTATTGTTATCCCTCCTTTTGGCGAATACCCC
GCTTTGACCCTTCACCCCTGGATATATGGGCAGCAGTACACCTTCTTCAGCATGGATGAACCAGGCAGTGA
GCAGTTCACGGTACTTGCAGACGTCCTCCTGAATAAGCCAGGCTTTGGCAACCGCTGCCTGAAGGAAGGGT
GGCTTCCGGAGTACCCCTGTGGCAACTCAACACCCTGGAAGACTCCTTCTGTGTCCCCAAACATCACCCAG
CTGTTCCAGAAGCAGAAATGGACACAGGTCAACCCTTCACCATCCTGCAGGTGCAGCACCAGGGAGAAGCT
CACCATGCTGCCAGAGTGCCCCGAGGGTGCCGGGGGCCTCCCGCCCCCCCAGAGAACACAGCGCAGCACGG
AAATTCTACAAGACCTGACGGACAGGAACATCTCCGACTTCTTGGTAAAAACGTATCCTGCTCTTATAAGA
AGCAGCTTAAAGAGCAAATTCTGGGTCAATGAACAGAGGTATGGAGGAATTTCCATTGGAGGAAAGCTCCC
AGTCGTCCCCATCACGGGGGAAGCACTTGTTGGGTTTTTAAGCGACCTTGGCCGGATCATGAATGTGAGCG
GGGGCCCTATCACTAGAGAGGCCTCTAAAGAAATACCTGATTTCCTTAAACATCTAGAAACTGAAGACAAC
ATTAAGGTGTGGTTTAATAACAAAGGCTGGCATGCCCTGGTCAGCTTTCTCAATGTGGCCCACAACGCCAT
CTTACGGGCCAGCCTGCCTAAGGACAGGAGCCCCGAGGAGTATGGAATCACCGTCATTAGCCAACCCCTGA
ACCTGACCAAGGAGCAGCTCTCAGAGATTACAGTGCTGACCACTTCAGTGGATGCTGTGGTTGCCATCTGC
GTGATTTTCTCCATGTCCTTCGTCCCAGCCAGCTTTGTCCTTTATTTGATCCAGGAGCGGGTGAACAAATC
CAAGCACCTCCAGTTTATCAGTGGAGTGAGCCCCACCACCTACTGGGTAACCAACTTCCTCTGGGACATCA
TGAATTATTCCGTGAGTGCTGGGCTGGTGGTGGGCATCTTCATCGGGTTTCAGAAGAAAGCCTACACTTCT
CCAGAAAACCTTCCTGCCCTTGTGGCACTGCTCCTGCTGTATGGATGGGCGGTCATTCCCATGATGTACCC
AGCATCCTTCCTGTTTGATGTCCCCAGCACAGCCTATGTGGCTTTATCTTGTGCTAATCTGTTCATCGGCA
TCAACAGCAGTGCTATTACCTTCATCTTGGAATTATTTGAGAATAACCGGACGCTGCTCAGGTTCAACGCC
GTGCTGAGGAAGCTGCTCATTGTCTTCCCCCACTTCTGCCTGGGCCGGGGCCTCATTGACCTTGCACTGAG
CCAGGCTGTGACAGATGTCTATGCCCGGTTTGGTGAGGAGCACTCTGCAAATCCGTTCCACTGGGACCTGA
TTGGGAAGAACCTGTTTGCCATGGTGGTGGAAGGGGTGGTGTACTTCCTCCTGACCCTGCTGGTCCAGCGC
CACTTCTTCCTCTCCCAATGGATTGCCGAGCCCACTAAGGAGCCCATTGTTGATGAAGATGATGATGTGGC
TGAAGAAAGACAAAGAATTATTACTGGTGGAAATAAAACTGACATCTTAAGGCTACATGAACTAACCAAGA
TTTATCCAGGCACCTCCAGCCCAGCAGTGGACAGGCTGTGTGTCGGAGTTCGCCCTGGAGAGTGCTTTGGC
CTCCTGGGAGTGAATGGTGCCGGCAAAACAACCACATTCAAGATGCTCACTGGGGACACCACAGTGACCTC
AGGGGATGCCACCGTAGCAGGCAAGAGTATTTTAACCAATATTTCTGAAGTCCATCAAAATATGGGCTACT
GTCCTCAGTTTGATGCAATCGATGAGCTGCTCACAGGACGAGAACATCTTTACCTTTATGCCCGGCTTCGA
GGTGTACCAGCAGAAGAAATCGAAAAGGTTGCAAACTGGAGTATTAAGAGCCTGGGCCTGACTGTCTACGC
CGACTGCCTGGCTGGCACGTACAGTGGGGGCAACAAGCGGAAACTCTCCACAGCCATCGCACTCATTGGCT
GCCCACCGCTGGTGCTGCTGGATGAGCCCACCACAGGGATGGACCCCCAGGCACGCCGCATGCTGTGGAAC
GTCATCGTGAGCATCATCAGAGAAGGGAGGGCTGTGGTCCTCACATCCCACAGCATGGAAGAATGTGAGGC
ACTGTGTACCCGGCTGGCCATCATGGTAAAGGGCGCCTTTCGATGTATGGGCACCATTCAGCATCTCAAGT
CCAAATTTGGAGATGGCTATATCGTCACAATGAAGATCAAATCCCCGAAGGACGACCTGCTTCCTGACCTG
AACCCTGTGGAGCAGTTCTTCCAGGGGAACTTCCCAGGCAGTGTGCAGAGGGAGAGGCACTACAACATGCT
CCAGTTCCAGGTCTCCTCCTCCTCCCTGGCGAGGATCTTCCAGCTCCTCCTCTCCCACAAGGACAGCCTGC
TCATCGAGGAGTACTCAGTCACACAGACCACACTGGACCAGGTGTTTGTAAATTTTGCTAAACAGCAGACT
GAAAGTCATGACCTCCCTCTGCACCCTCGAGCTGCTGGAGCCAGTCGACAAGCCCAGGACTGAAAGCTTAT
CGATAATCAACCTCTGGATTACAAAATTTGTGAAAGATTGACTGGTATTCTTAACTATGTTGCTCCTTTTA
CGCTATGTGGATACGCTGCTTTAATGCCTTTGTATCATGCTATTGCTTCCCGTATGGCTTTCATTTTCTCC
TCCTTGTATAAATCCTGGTTGCTGTCTCTTTATGAGGAGTTGTGGCCCGTTGTCAGGCAACGTGGCGTGGT
GTGCACTGTGTTTGCTGACGCAACCCCCACTGGTTGGGGCATTGCCACCACCTGTCAGCTCCTTTCCGGGA
CTTTCGCTTTCCCCCTCCCTATTGCCACGGCGGAACTCATCGCCGCCTGCCTTGCCCGCTGCTGGACAGGG
GCTCGGCTGTTGGGCACTGACAATTCCGTGGTGTTGTCGGGGAAATCATCGTCCTTTCCTTGGCTGCTCGC
CTGTGTTGCCACCTGGATTCTGCGCGGGACGTCCTTCTGCTACGTCCCTTCGGCCCTCAATCCAGCGGACC
TTCCTTCCCGCGGCCTGCTGCCGGCTCTGCGGCCTCTTCCGCGTCTTCGCCTTCGCCCTCAGACGAGTCGG
ATCTCCCTTTGGGCCGCCTCCCCGCATGCCGCTGATCAGCCTCGACTGTGCCTTCTAGTTGCCAGCCATCT
GTTGTTTGCCCCTCCCCCGTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTGGGGTGGGGCAGGACAGCA
AGGGGGAGGATTGGGAAGACAATAGCAGGCATGCTGGGGATGCGGTGGGCTCTATGGCTTCTGAGGCGGAA
AGAACCAGCTGGGG

### EXAMPLES

### Example 1—Preparation of Upstream and Downstream AAV Vectors

For generation of a given AAV vector, three plasmids are required: pTransgene, pRepCap and pHelper. pTransgene contains either the upstream or downstream ABCA4 transgene as detailed below (ITR integrity confirmed). pRepCap contains the rep and cap genes of the AAV genome. The rep genes are from the AAV2 genome whereas the cap genes will vary depending on serotype requirement. pHelper contains the required adenoviral genes necessary for successful AAV generation. The plasmids are complexed with polyethylenimine (PEI) for a triple transfection mix that is applied to HEK293T cells. Three days post-transfection, the cells are collected and lysed. The lysate is treated with Benzonase and clarified before applying to an iodixanol gradient comprised of 15%, 25%, 40% and 60% phases. The gradients are spun at 59,000 rpm for 1 hour 30 minutes and the 40% fraction is then withdrawn. This AAV phase is then purified and concentrated using an Amicon Ultra 100K filter unit. Following this step, 100-200 μl of purified AAV is obtained in PBS.

### Example 2—Structure of Example AAV Vectors

### Upstream Vector

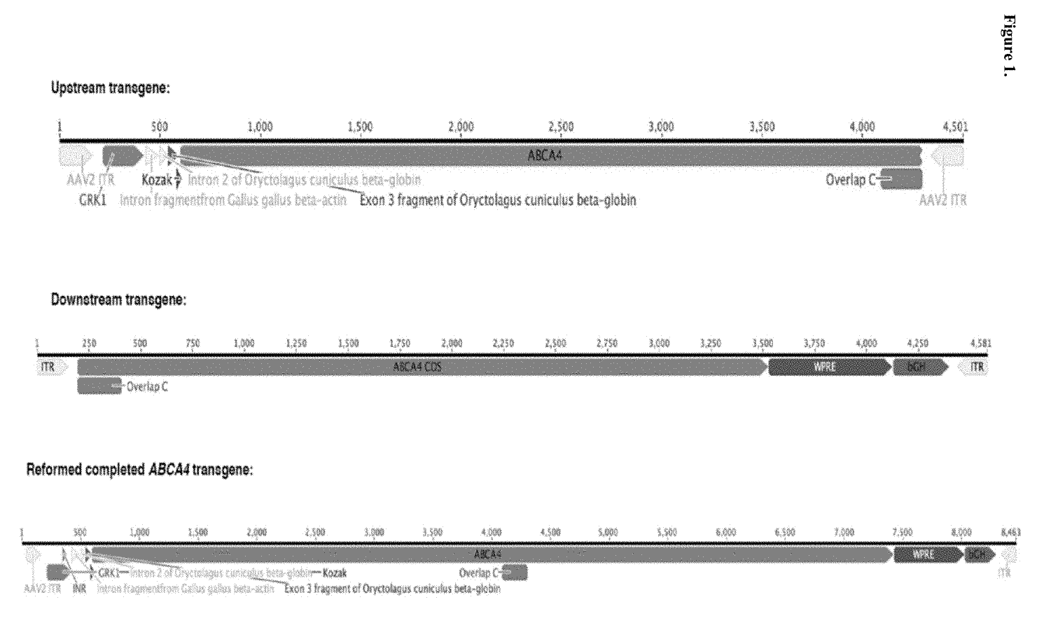

This vector contains a promoter, untranslated region (UTR) and upstream segment of ABCA4 CDS with an AAV2 ITR at each end of the transgene (FIG. 1). ABCA4 is expressed in photoreceptor cells of the retina and therefore a human rhodopsin kinase (GRK1) promoter element has been incorporated. The specific GRK1 promoter sequence contained in the upstream vector is as described by Khani et al. (Investigative Ophthalmology and Visual Science, 48(9), 3954-3961, 2007) comprising of nucleotides −112 to +87 of the GRK1 gene and has been used in pre-clinical studies for gene therapy targeting the photoreceptor cells.

The 199 nucleotides of the GRK1 promoter are followed by an untranslated region (UTR) 186 nucleotides in length. This nucleotide sequence was selected from the larger UTR (443 nucleotides) contained in the REP1 clinical trial vector (MacLaren et al., 2014). Specifically, the selected sequence includes a Gallus gallus β-actin (CBA) intron 1 fragment (with predicted splice donor site), Oryctolagus cuniculus β-globin (RBG) intron 2 fragment (including predicted branch point and splice acceptor site) and Oryctolagus cuniculus β-globin exon 3 fragment immediately prior to the Kozak consensus, which leads into the ABCA4 CDS. This UTR fragment has been added to the original GRK1 promoter element to increase translational yield (Rafiq et al., 1997; Chatterjee et al., 2009). By itself, the GRK1 promoter has shown very good gene expression capabilities in photoreceptor cells, suggesting there are no major features inhibiting expression.

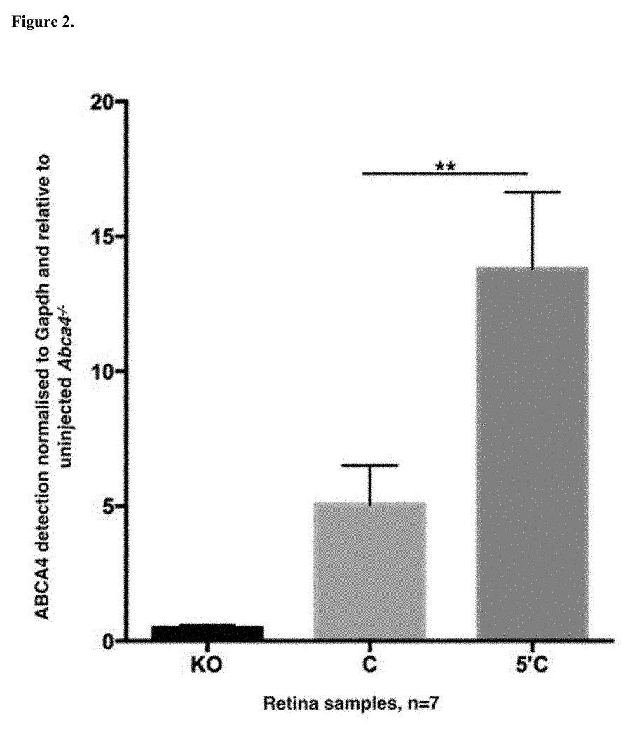

Comparison of dual vector injected Abca4^{-/-} retinae reveals significantly more ABCA4 protein is generated from eyes in which the upstream vector carries the GRK1.5′UTR element compared to the GRK1 promoter element alone (FIG. 2).

Following the Kozak consensus in the upstream vector is the ABCA4 CDS from nucleotide 1 to 3,701 (105 to 3,805 in NCBI reference file NM_000350). The final 208 nucleotides of the ABCA4 CDS form the first 208 nucleotides of CDS contained in the downstream vector and serve as the overlap zone. The coding sequence fragment contained in the upstream vector matches the reference sequence NM_000350 with the exception of a base change at nucleotide 1,536 (NM_000350 1,640) G>T. This is the third base of the codon and does not result in an amino acid sequence change. The ABCA4 CDS is truncated within exon 25 with the 3′ITR downstream of this.

### Downstream Vector

This vector contains the downstream segment of ABCA4 CDS, a Woodchuck hepatitis virus post-transcriptional response element (WPRE) and bovine growth hormone poly-adenylation signal (bGH polyA) with an AAV2 ITR at each end of the transgene (FIG. 1). The ABCA4 CDS begins downstream of the 5′ITR at position 3,494 (NM_000350 3,598) and continues to the stop codon at 6,822 (NM_000350 6,926). The first 208 nucleotides of the ABCA4 CDS are the same as the final 208 ABCA4 CDS nucleotides contained in the upstream vector and serve as the overlap zone between transgenes. The coding sequence fragment contained in the downstream vector matches the reference sequence NM_000350 with the exception of a base change at nucleotide 5,175 (NM_000350 5,279) G>A and 6,069 (NM_000350 6,173) T>C. These changes both occur in the third base of a codon and do not result in an amino acid sequence change.

The restriction site HindIII separates the ABCA4 CDS stop codon from the WPRE. This element is 593 nucleotides in length and matches the X antigen inactivated WPRE contained in the REP1 clinical trial vector. A restriction site for SphI then separates the WPRE from the bGH poly A signal, which is 269 nucleotides in length and matches the bGH poly A signal present in the REP1 clinical trial vector. The 3′ITR then lies downstream of the polyA signal.

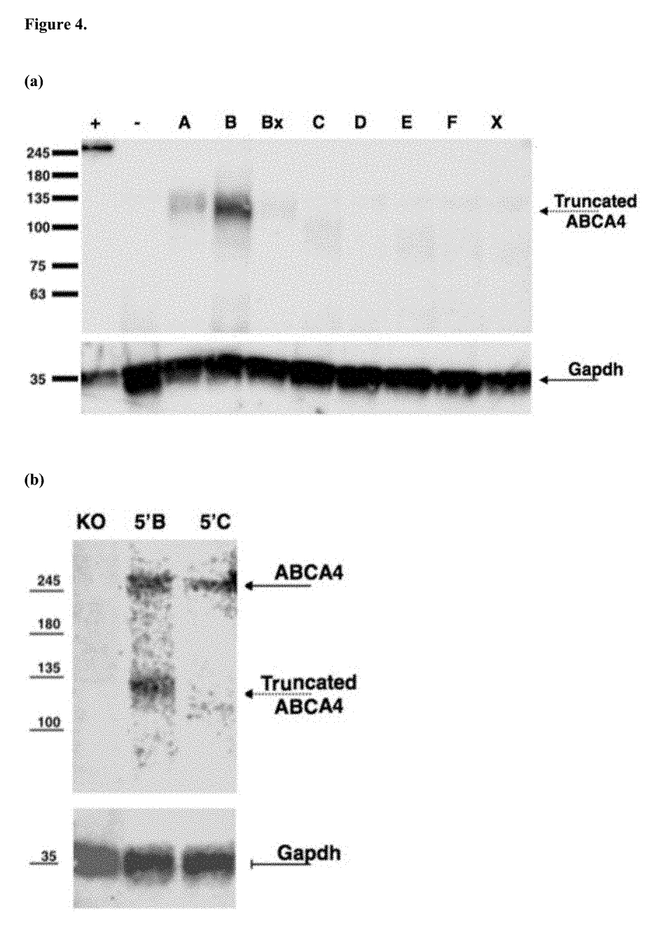

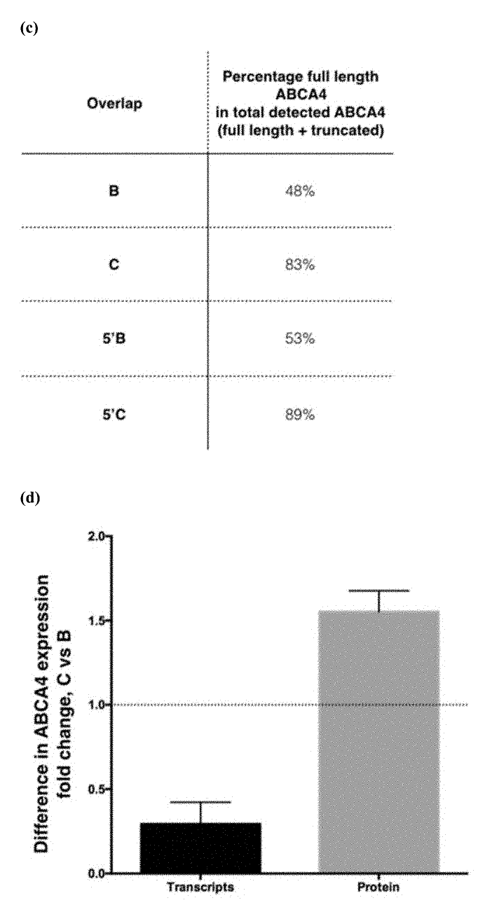

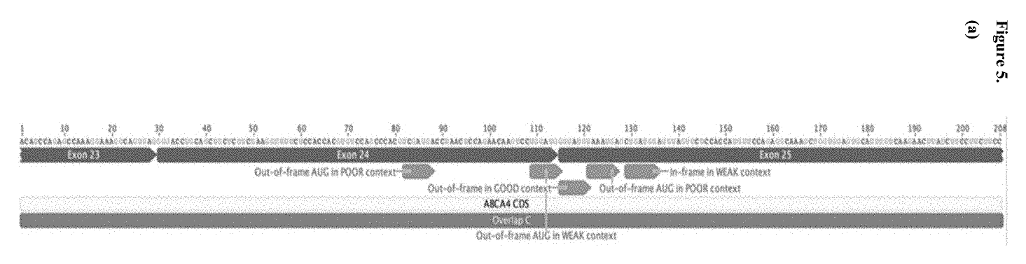

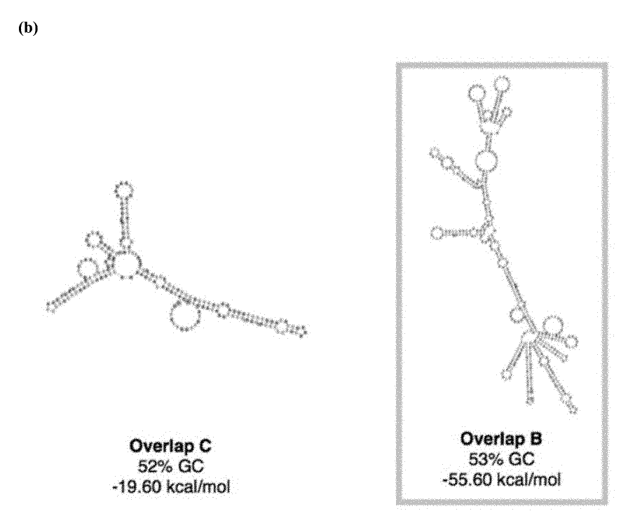

The AAV2 5′ITR is known to have promoter activity and with the WPRE and bGH poly A signal within the downstream transgene, stable transcripts will be generated from unrecombined downstream vectors. The wild-type ABCA4 CDS contained in the downstream transgene carries multiple in-frame AUG codons that cannot be substituted for other codons without altering the amino acid sequence. This creates the possibility of translation occurring from the stable transcripts, leading to the presence of truncated ABCA4 peptides that are detectable by western blot (FIG. 4a). The starting sequence of the chosen overlap zone was carefully selected to include an out-of-frame AUG codon in good context (regarding potential Kozak consensus) prior to an in-frame AUG codon in weaker context (FIG. 5a) in order to encourage the translational machinery to initiate from an out-of-frame site. There are in total four out-of-frame AUG codons in various contexts prior to the in-frame AUG. All of these would translate to a STOP codon within 10 amino acids. The existence of these out-of-frame AUG codons may prevent translation of truncated ABCA4 proteins from unrecombined downstream transgenes.

### Example 3—Assessment of Overlap Zones

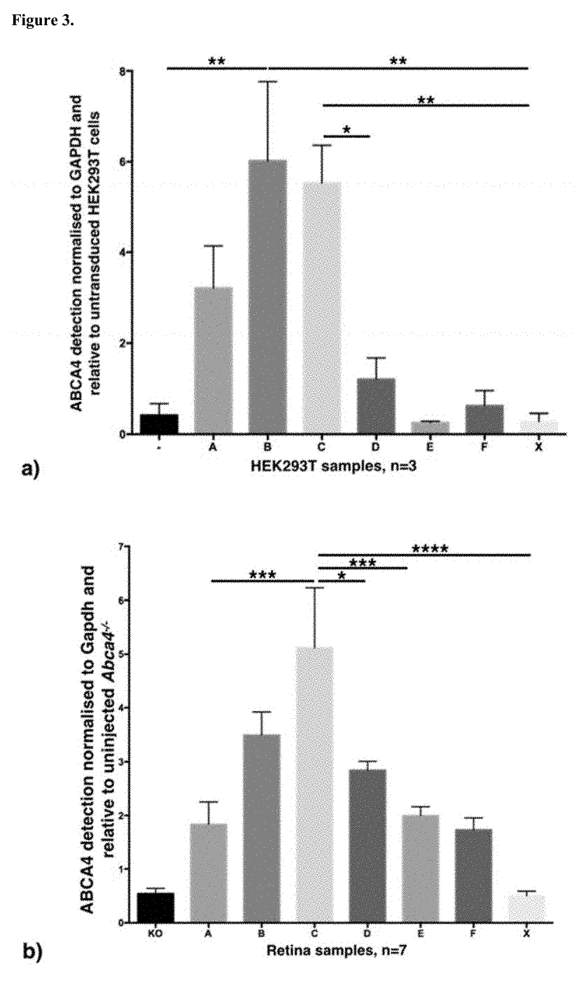

The optimal overlap zone was determined following in vitro and in vivo assessments of six overlap variants (FIG. 3a & 3b, respectively). These are referred to as A, B, C, D, E and F and represent the following overlap zones (X represents no overlap): A. 1,173 nucleotides (3259-4430); B. 506 nucleotides (3300-3805); C. 208 nucleotides (3598-3805); D. 99 nucleotides (3707-3805); E. 49 nucleotides (3757-3805) and; F. 24 nucleotides (3782-3805). Downstream transgenes for overlap zones B to X are all paired with the same upstream transgene. Overlap variants B and C performed better than all other variants and to a similar extent but dual vector version C was selected for various reasons. The first is due to its limited production of truncated ABCA4 from unrecombined downstream transgenes (FIG. 4a). The unrecombined downstream transgenes from C, D, E, F and X variants generate significantly reduced levels of truncated ABCA4 protein than the A or B versions. In a dual vector context, overlap C generates the lowest proportion of truncated ABCA4 compared to full length ABCA4 (FIGS. 4b and 4c). This suggests the overlap C transgene design is not only limiting unwanted expression from unrecombined transgenes but is also recombining with greater efficiency than the overlap B. Further evidence of this arises by comparing transcript fold change and protein fold change differences between overlap C and B injected ABCA4^{-/-} retinae. Primers targeting the upstream portion of ABCA4 CDS (therefore detecting transcripts from unrecombined upstream transgenes in addition to full length ABCA4 transcripts from recombined transgenes) detected very high levels of transcripts present in both overlap B and C dual vector injected retinae. However, overlap C generated less than half the transcript levels of overlap B yet produced 1.5 times the level of ABCA4 protein (FIG. 4d). Given that both share the same upstream vector and differ only in their downstream transgene sequence, this suggests the overlap zone selected for overlap C recombines with greater efficiency than overlap B.

The overlap zone selected has a GC content of 52% and free energy prediction of −19.60 kcal/mol, which is nearly three times less that of overlap zone B at −55.60 kcal/mol (53% GC content), FIG. 5b. This reduction in free energy suggests a secondary structure formed by unrecombined overlap C will be easier to resolve than for overlap B, which we predict leaves it more available for interaction with the overlap zone on the opposing transgene.

### Example 4—Experimental Protocols

### FIG. 2

Abca4^{-/-} mice received a 2 μl subretinal injection of a dual vector mix (1:1), delivering 1E+9 genome copies of each vector per eye. Enucleation of the eye was performed 6 weeks post-injection with the neural retina dissected from the eye cup and lysed in RIPA buffer. The tissue was homogenised and the supernatant extracted following centrifugation. Supernatants were mixed with denaturing loading buffer and run on a 7.5% TGX gel under denaturing conditions. Proteins were transferred to a PVDF membrane and ABCA4 detected with rabbit polyclonal anti-ABCA4 (Abcam) and Gapdh detected with mouse monoclonal anti-GAPDH (Origene). Bands were visualised and analysed using the LICOR imaging system. ABCA4 levels were normalised to Gapdh for each sample and then represented relative to uninjected Abca4^{-/-} eyes.

### FIG. 3a

HEK293T cells were used to seed 6 well culture plates at 2E5 cells per well. After 24 hours, one well of cells was lifted and counted. This count was used to determine the appropriate amount of vector to provide to each well to give a multiplicity of infection (MOI) of 10,000 per vector. The culture media was removed and the AAV added in 1 ml of media containing no foetal bovine serum (FBS). Cells were incubated for one hour at 37° C. before adding 1 ml of media containing 20% FBS. 48 hours post-transduction the media was removed and fresh media containing 10% FBS applied. Cells were incubated for a further 48 hours after which another media change occurred. 24 hours later, cells were harvested and washed three times in cold PBS using a gentle centrifugation cycle. The final PBS wash was removed and the cell pellets frozen. Cell pellets were thawed on ice then lysed in RIPA buffer. Lysates were treated as per the retina samples described above for western blot analysis.

### FIG. 3b

As for FIG. 2.

### FIG. 4a

HEK293T cells were used to seed 6 well culture plates at 1E6 cells per well. After 24 hours, a transfection mix containing 1 μg of plasmid complexed to transfection reagent LT1 (GeneFlow) was applied to the cells. Test plasmids carried the downstream transgenes used in the creation of AAV vectors. 48 hours post-transfection, cells were washed, harvested and assessed by western blot as described above.

### FIG. 4b

As for FIG. 2

### FIG. 4d

ABCA4 protein levels were obtained from western blot analyses as described in FIG. 2 and the fold change compared between overlap variant C and B dual vector treatments. For transcript level comparisons, tissue samples were collected in RNAlater (Ambion) and the mRNA extracted using Dynabeads-oligodT mRNA DIRECT (Life Technologies). cDNA synthesis was performed with 500 ng mRNA using an oligodT primer and SuperScript III (Life Technologies). Samples were cleaned using PCR Purification Spin Columns (QIAGEN) and eluted in 50 μl DEPC-treated water. The cDNA was assessed by qPCR targeting an upstream portion of the ABCA4 CDS. Levels of ABCA4 were normalised to Actin levels and expressed relative to uninjected Abca4^{-/-} samples. The fold change in ABCA4 transcript levels between overlap variant C and B dual vector treatments were then compared.

### Example 5—AAV-Mediated Delivery of ABCA4 to the Photoreceptors of Abca4^{-/-} Mice Using an Overlapping Dual Vector Strategy

The data presented in this Example demonstrate the expression of ABCA4 protein specifically localised in the photoreceptor outer segments of the Abca4^{-/-} mouse model following sub retinal injection with an overlapping dual vector system of the invention.

### Transgene Design and Production:

Overlapping ABCA4 transgenes were packaged into AAV8 Y733F capsids. The upstream transgene contained the human rhodopsin kinase (GRK1) promoter and an upstream portion of the ABCA4 coding sequence (CDS) between AAV2 inverted terminal repeats (ITRs). The downstream transgene contained a downstream portion of the ABCA4 CDS, Woodchuck hepatitis virus post-transcriptional regulatory element (WPRE) and a polyA signal (pA). Both the upstream and downstream transgenes carried a region of ABCA4 CDS overlap.

### Injections:

Abac4−/− mice received a 2 μl sub retinal injection at 4-5 weeks of age containing a 1:1 mix of the upstream and downstream vectors (1×10¹³ gc/ml). Eyes were harvested at 6 weeks post-injection for immunohistochemical (IHC) assessments.

### Immunohistochemical Staining:

Whole eye cups with the lens removed were fixed in 4% paraformaldehyde (PFA) for 20 minutes then incubated in 30% sucrose overnight at 4° C. Eyes were frozen in mounting medium before being sectioned. Tissue slices were dried overnight at room temperature then rinsed in phosphate buffered saline (PBS) for 5 minutes, three times. Samples were permeabilised with 0.2% Triton-X-100 for 20 minutes then washed three times in PBS before incubating with 10% donkey serum (DS), 1% bovine serum albumin (BSA), 0.1% Triton-X-100 for one hour. Antibodies were diluted 1/200 in 1% DS, 0.1% BSA, applied to sections and left for two hours at room temperature. Abca4/ABCA4 detection was achieved with goat anti-ABCA4 (AntibodiesOnline), hyperpolarisation activated cyclic nucleotide gated potassium channel 1 (Hcn1) detection with mouse anti-Hcn1 (Abcam) and rhodopsin detection with mouse anti-1D4 (Abcam). Sections were rinsed three times with 0.05% Tween-20 then secondary antibodies applied (diluted 1/400) for one hour under dark conditions. Sections were rinsed twice with 0.05% Tween-20 then incubated with Hoescht stain (1/1,000) for 15 minutes. Sections were rinsed in PBS then leg to air dry. Diamond anti-fade mounting medium was applied to each section and slides were left overnight before imaging.

### Results:

### ABCA4 Expression Localised to Photoreceptor Cell Outer Segments.

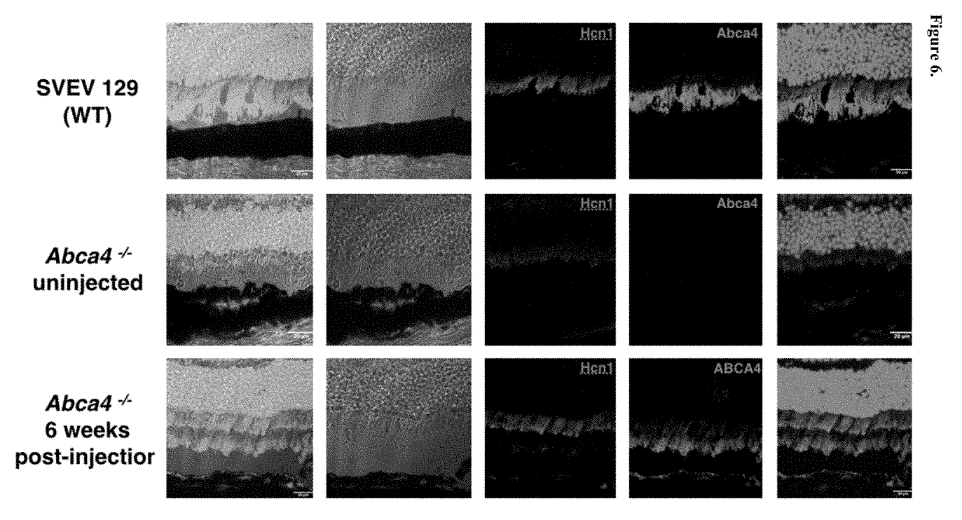

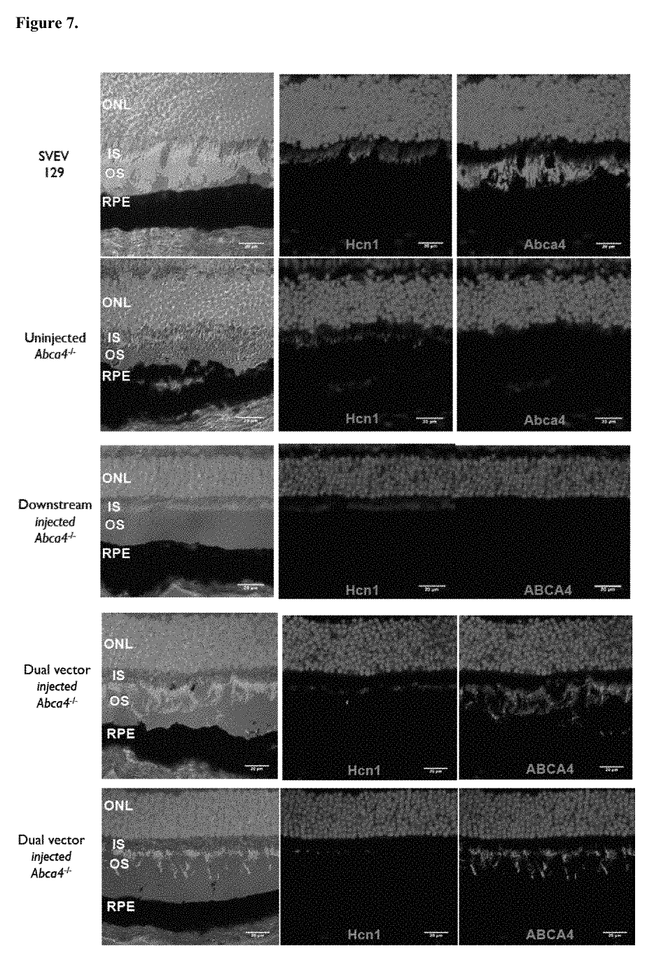

FIG. 7 shows Abca4/ABCA4 (green) and Hcn1 (red) staining in wild-type (WT) and Abca4^{-/-} eyes. WT SVEV 129, uninjected and injected Abca4^{-/-} eyes were stained for the photoreceptor inner segment marker Hcn1 and Abca4/ABCA4. WT and dual vector treated Abca4^{-/-} eyes revealed specific localisation of Abca4/ABCA4 in the photoreceptor cell outer segments.

ABCA4 Co-Localisation with Rhodopsin.

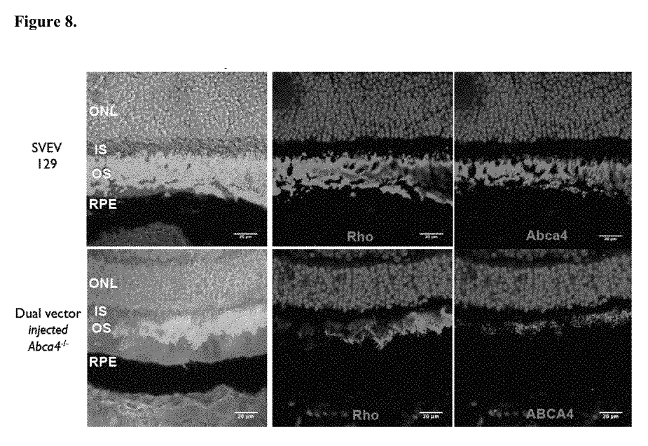

FIG. 8 shows Abca4/ABCA4 (green) and rhodopsin (red) staining in photoreceptor cell outer segments in wild-type (WT) and Abca4^{-/-} eyes. WT and dual vector treated Abca4^{-/-} eyes revealed colocalisation of rhodopsin and Abca4/ABCA4 in the photoreceptor cell outer segments.

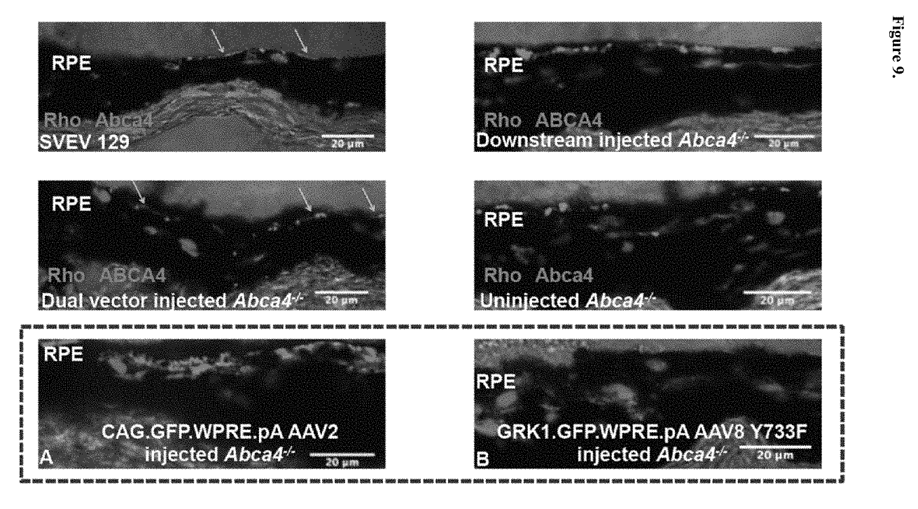

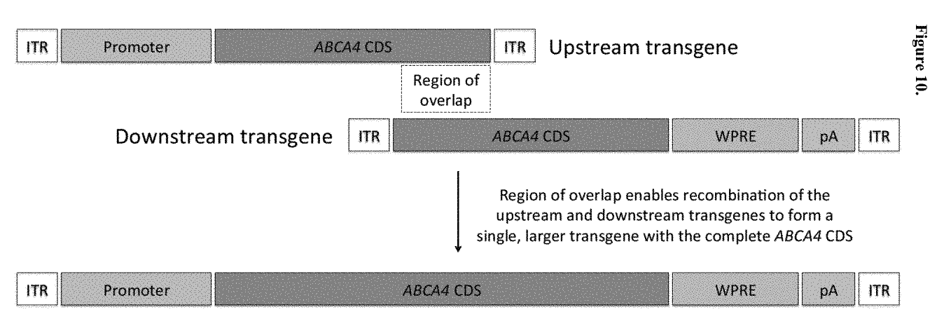

FIG. 9 shows Abca4/ABCA4 (green) and rhodopsin (red) apical RPE staining in wild-type (WT) and Abca4^{-/-} eyes. WT and dual vector treated Abca4^{-/-} eyes revealed co-localisation of rhodopsin and Abca4/ABCA4 in the apical regions of RPE cells, hypothesised to originate from shed outer segment discs. Abca4^{-/-} eyes not treated with the dual vector showed only rhodopsin staining in the apical region of RPE cells. Boxed image A shows the expression pattern achieved from transduced RPE cells, revealing a diffuse staining pattern in contrast to the Abca4/ABCA4/rho staining. Image B confirms no RPE expression from the GRK1 promoter.

### Conclusions:

An optimised overlapping dual vector system can be used to generate ABCA4 expression in photoreceptor cells where it is trafficked to the desired outer segment structures at levels detectable by IHC.

### Example 6—Bisretinoid/A2E Assessments in Dual Vector Treated Abca4^{-/-} Mice

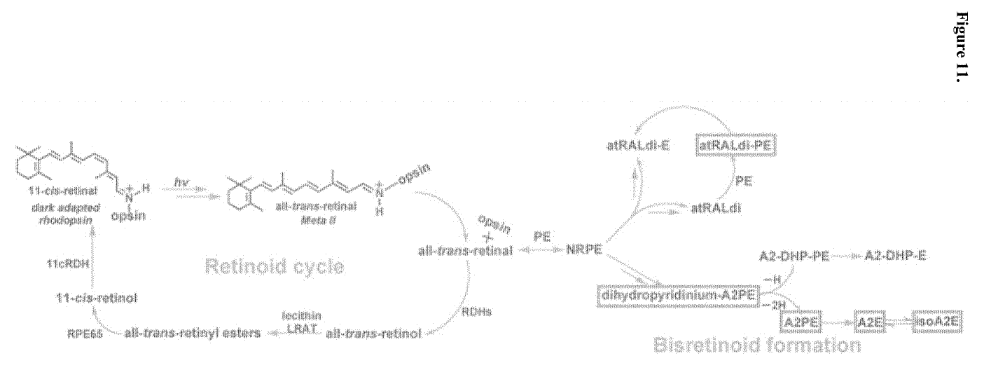

The Abca4^{-/-} mouse model exhibits an increase with age in levels of bisretinoids and A2E compared to wildtype mice. In contrast to humans, however, the increase in bisretinoids does not reach a level that would be required to cause any significant retinal degeneration. This suggests that other compensatory mechanisms may exist in the Abca4 deficient mouse eye. In a wildtype retina. Abca4 facilitates the movement of retinal out of the photoreceptor cell outer segment disc membranes for recycling. When there is an absence of functional Abca4, as in the Abca4^{-/-} mouse model, the retinal is maintained in the outer segment disc membranes where it undergoes biochemical changes into various bisretinoid forms (FIG. 11). Photoreceptor cells constantly generate new outer segment discs and in doing so there is movement of the older more distal discs towards the RPE cells, which subsequently degrade them by phagocytosis. In the Abca4 deficient mouse the phagocytosed discs contain elevated levels of bisretinoids. Within the RPE cells these are further converted into A2E isoforms, the accumulation of which leads to lipofuscin. Hence although the bisretinoid accumulation in the Abca4 deficient mouse is insufficient to cause a retinal degeneration, the resulting elevated levels above baseline may nevertheless be quantified and thus provide a biomarker of Abca4 function.

Bisretinoid and A2E compounds can be accurately measured by high-performance liquid chromatography (HPLC). A measure of therapeutic efficacy in mice treated with ABCA4 gene therapy would therefore be to achieve a reduction in the levels of bisretinoids and A2E compared to untreated eyes. There are however two considerations that need to be addressed. In the first instance, for clinical application we need to use a human ABCA4 coding sequence and a human photoreceptor promoter and this is unlikely to be as efficacious in the mouse. Furthermore HPLC measurements are taken from the whole eye and not just the region exposed to the vector by the subretinal injection. Hence the overall reduction in bisretoids in the Abca4 deficient mouse is unlikely to reach wildtype levels. The second consideration is the subretinal injection, which may lead to damage of the outer segment discs. Since these structures are rich in bisretinoids, the effects of ABCA4 gene therapy need to be compared with a similar sham injection. Ideally the contralateral eye of the same mouse should be used for this to control for eye size and lifetime light exposure, which may also influence bisretinoid accumulation.

For this reason we compared the bisretinoid/A2E levels in a cohort of Abca4^{-/-} mice that received a sham injection in one eye and a similar treatment injection in the contralateral eye. Each sham eye received the upstream vector at the same total AAV dose as that which was received in the paired dual vector treatment eye. Both eyes of each mouse therefore received a 2 μl subretinal injection, forming a bleb containing 2×10¹⁰ genome particles of AAV vector.

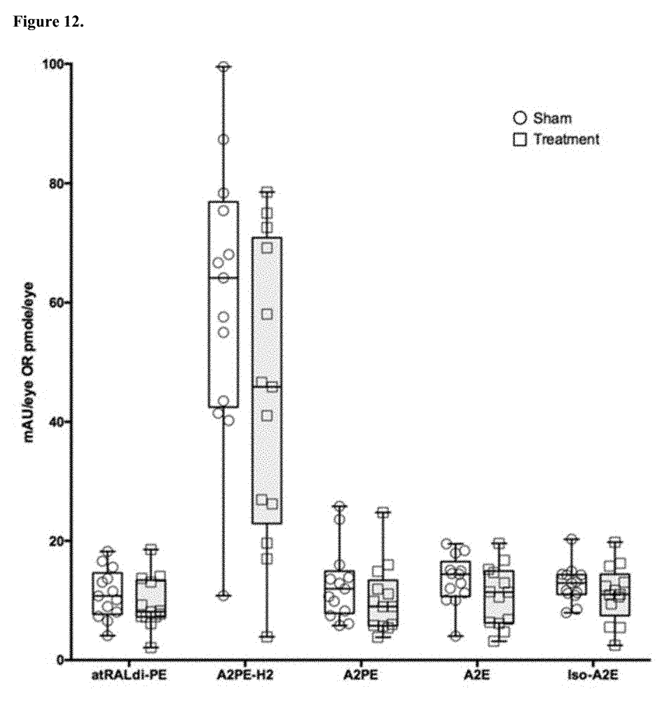

A total of 13 Abca4 knockout mice were injected at 4-5 weeks of age and eyes were harvested 3 months post-injection. Mice were dark adapted for 16 hours prior to tissue collection, which was conducted in the dark under dim red light. Whole eyes were then anonymized and shipped frozen to the Jules Stein Eye Institute for bisretinoid/A2E assessments using established HPLC assays. Each whole eye was taken and processed without dissection. Following HPLC assessments of all 26 eyes, the identities were subsequently unmasked and bisretinoid/A2E levels for each treated eye were compared to their paired sham injected eye. Two-way ANOVA determined the treatment to have a significant effect on the levels of bisretinoid/A2E with a reduction in dual vector treated eyes observed compared to paired sham injected eyes (p=0.0171), FIG. 12.

All publications mentioned in the above specification are herein incorporated by reference. Various modifications and variations of the described products, systems, uses, processes and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention, which are obvious to those skilled in biochemistry and biotechnology or related fields, are intended to be within the scope of the following claims.

1. An adeno-associated viral (AAV) vector system for expressing a human ABCA4 protein in a target cell, the AAV vector system comprising a first AAV vector comprising a first nucleic acid sequence and a second AAV vector comprising a second nucleic acid sequence;
wherein the first nucleic acid sequence comprises a 5′ end portion of an ABCA4 coding sequence (CDS) and the second nucleic acid sequence comprises a 3′ end portion of an ABCA4 CDS, and the 5′ end portion and the 3′ end portion together encompass the entire ABCA4 CDS;
wherein the first nucleic acid sequence comprises a sequence of contiguous nucleotides corresponding to nucleotides 105 to 3597 of SEQ ID NO: 1;
wherein the second nucleic acid sequence comprises a sequence of contiguous nucleotides corresponding to nucleotides 3806 to 6926 of SEQ ID NO: 1;
wherein the first nucleic acid sequence and the second nucleic acid sequence each comprise a region of sequence overlap with the other; and
wherein the region of sequence overlap comprises at least about 20 contiguous nucleotides of a nucleic acid sequence corresponding to nucleotides 3598 to 3805 of SEQ ID NO: 1.

2. The AAV vector system of claim 1, wherein the region of sequence overlap is between 20 and 550 nucleotides in length; preferably between 50 and 250 nucleotides in length; preferably between 175 and 225 nucleotides in length; preferably between 195 and 215 nucleotides in length.

3. The AAV vector system of claim 1 or claim 2, wherein the region of sequence overlap comprises at least about 50 contiguous nucleotides of a nucleic acid sequence corresponding to nucleotides 3598 to 3805 of SEQ ID NO: 1; preferably at least about 75 contiguous nucleotides; preferably at least about 100 contiguous nucleotides; preferably at least about 150 contiguous nucleotides; preferably at least about 200 contiguous nucleotides; preferably all 208 contiguous nucleotides.

4. The AAV vector system of any preceding claim,
wherein the first nucleic acid sequence comprises a sequence of contiguous nucleotides corresponding to nucleotides 105 to 3805 of SEQ ID NO: 1; and
wherein the second nucleic acid sequence comprises a sequence of contiguous nucleotides corresponding to nucleotides 3598 to 6926 of SEQ ID NO: 1.

5. The AAV vector system of any preceding claim, wherein the first nucleic acid sequence comprises a GRK1 promoter operably linked to the 5′ end portion of an ABCA4 coding sequence (CDS).

6. The AAV vector system of any preceding claim, wherein the first nucleic acid sequence comprises an untranslated region (UTR) located upstream of the 5′ end portion of an ABCA4 coding sequence (CDS).

7. The AAV vector system of any preceding claim, wherein the second nucleic acid sequence comprises a post-transcriptional response element (PRE); preferably a Woodchuck hepatitis virus post-transcriptional response element (WPRE).

8. The AAV vector system of any preceding claim, wherein the second nucleic acid sequence comprises a bovine Growth Hormone (bGH) poly-adenylation sequence.

9. The AAV vector system of any preceding claim, wherein the first AAV vector comprises the nucleic acid sequence of SEQ ID NO: 9; and wherein the second AAV vector comprises the nucleic acid sequence of SEQ ID NO: 10.

10. A method for expressing a human ABCA4 protein in a target cell, the method comprising the steps of:
transducing the target cell with the first AAV vector and the second AAV vector as defined in any of claims 1-9, such that a functional ABCA4 protein is expressed in the target cell.

11. An AAV vector comprising a nucleic acid sequence comprising a 5′ end portion of an ABCA4 CDS, wherein the 5′ end portion of an ABCA4 CDS consists of a sequence of contiguous nucleotides corresponding to nucleotides 105 to 3805 of SEQ ID NO: 1.

12. The AAV vector of claim 11, wherein the AAV vector comprises the nucleic acid sequence of SEQ ID NO: 9.

13. An AAV vector comprising a nucleic acid sequence comprising a 3′ end portion of an ABCA4 CDS, wherein the 3′ end portion of an ABCA4 CDS consists of a sequence of contiguous nucleotides corresponding to nucleotides 3598 to 6926 of SEQ ID NO: 1.

14. The AAV vector of claim 13, wherein the AAV vector comprises the nucleic acid sequence of SEQ ID NO: 10.

15. A nucleic acid comprising the first nucleic acid sequence as defined in any one of claims 1 to 9.

16. A nucleic acid comprising the second nucleic acid sequence as defined in any one of claims 1 to 9.

17. A nucleic acid comprising the nucleic acid sequence of SEQ ID NO: 9.

18. A nucleic acid comprising the nucleic acid sequence of SEQ ID NO: 10.

19. A kit comprising the first AAV vector as defined in any of claims 1 to 9 and the second AAV vector as defined in any of claims 1 to 9.

20. A kit comprising the nucleic acid of claim 15 and the nucleic acid of claim 16, or the nucleic acid of claim 17 and the nucleic acid of claim 18.

21. A pharmaceutical composition comprising the AAV vector system of any of claims 1 to 9 and a pharmaceutically acceptable excipient.

22. An AAV vector system according to any of claims 1-9, a kit according to claim 19 or claim 20, or a pharmaceutical composition according to claim 21, for use in gene therapy.

23. An AAV vector system according to any of claims 1-9, a kit according to claim 19 or claim 20, or a pharmaceutical composition according to claim 21, for use in preventing or treating disease characterised by degradation of retinal cells; preferably for use in preventing or treating Stargardt disease.

24. A method for preventing or treating a disease characterised by degradation of retinal cells, preferably Stargardt disease, comprising administering to a subject in need thereof an effective amount of an AAV vector system according to any of claims 1-9, a kit according to claim 19 or claim 20, or a pharmaceutical composition according to claim 21.


SEQUENCE LISTING (11 sequences)

SEQ ID NO: 1 - DNA, 7326 bp, Homo sapiens
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgcctggtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtgaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaattgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 2 - DNA, 7326 bp, Homo sapiens
aggacacagcgtccggagccagaggcgctcttaacggcgtttatgtcctttgctgtctgaggggcctcagctctgaccaatctggtcttcgtgtggtcattagcatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgatctttcacaccgctcgttcctgcagccagaaaggaactctgggcagctggaggcgcaggagcctgtgcccatatggtcatccaaatggactggccagcgtaaatgaccccactgcagcagaaaacaaacacacgaggagcatgcagcgaattcagaaagaggtctttcagaaggaaaccgaaactgacttgctcacctggaacacctgatggtgaaaccaaacaaatacaaaatccttctccagaccccagaactagaaaccccgggccatcccactagcagctttggcctccatattgctctcatttcaagcagatctgcttttctgcatgtttgtctgtgtgtctgcgttgtgtgtgattttcatggaaaaataaaatgcaaatgcactcatcacaaa

SEQ ID NO: 3 - DNA, 4464 bp, Artificial Sequence
  example upstream vector sequence, comprising ITR, promoter, CDS, ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaatggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccatttaaattagggataacagggtaatggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 4 - DNA, 4581 bp, Artificial Sequence
  example downstream vector sequence, comprising ITR, CDS, post-transcriptional response element, poly-adenylation sequence, ITR
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggcaattcagtcgataactataacggtcctaaggtagcgatttaaataacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggatttaaattagggataacagggtaatggcgcgggccgcaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 5 - DNA, 199 bp, Homo sapiens
gggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccggg

SEQ ID NO: 6 - DNA, 186 bp, Artificial Sequence
  untranslated region (UTR) sequence
gtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgg

SEQ ID NO: 7 - DNA, 593 bp, Woodchuck hepatitis virus
atcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctcccc

SEQ ID NO: 8 - DNA, 269 bp, Artificial Sequence
  bovine Growth Hormone poly-adenylation sequence
cgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 9 - DNA, 4087 bp, Artificial Sequence
  example partial upstream vector sequence, comprising promoter, CDS
ggtaccgggccccagaagcctggtggttgtttgtccttctcaggggaaaagtgaggcggccccttggaggaaggggccgggcagaatgatctaatcggattccaagcagctcaggggattgtctttttctagcaccttcttgccactcctaagcgtcctccgtgaccccggctgggatttagcctggtgctgtgtcagccccgggtgccgcagggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattaccaccatgggcttcgtgagacagatacagcttttgctctggaagaactggaccctgcggaaaaggcaaaagattcgctttgtggtggaactcgtgtggcctttatctttatttctggtcttgatctggttaaggaatgccaacccgctctacagccatcatgaatgccatttccccaacaaggcgatgccctcagcaggaatgctgccgtggctccaggggatcttctgcaatgtgaacaatccctgttttcaaagccccaccccaggagaatctcctggaattgtgtcaaactataacaactccatcttggcaagggtatatcgagattttcaagaactcctcatgaatgcaccagagagccagcaccttggccgtatttggacagagctacacatcttgtcccaattcatggacaccctccggactcacccggagagaattgcaggaagaggaatacgaataagggatatcttgaaagatgaagaaacactgacactatttctcattaaaaacatcggcctgtctgactcagtggtctaccttctgatcaactctcaagtccgtccagagcagttcgctcatggagtcccggacctggcgctgaaggacatcgcctgcagcgaggccctcctggagcgcttcatcatcttcagccagagacgcggggcaaagacggtgcgctatgccctgtgctccctctcccagggcaccctacagtggatagaagacactctgtatgccaacgtggacttcttcaagctcttccgtgtgcttcccacactcctagacagccgttctcaaggtatcaatctgagatcttggggaggaatattatctgatatgtcaccaagaattcaagagtttatccatcggccgagtatgcaggacttgctgtgggtgaccaggcccctcatgcagaatggtggtccagagacctttacaaagctgatgggcatcctgtctgacctcctgtgtggctaccccgagggaggtggctctcgggtgctctccttcaactggtatgaagacaataactataaggcctttctggggattgactccacaaggaaggatcctatctattcttatgacagaagaacaacatccttttgtaatgcattgatccagagcctggagtcaaatcctttaaccaaaatcgcttggagggcggcaaagcctttgctgatgggaaaaatcctgtacactcctgattcacctgcagcacgaaggatactgaagaatgccaactcaacttttgaagaactggaacacgttaggaagttggtcaaagcctgggaagaagtagggccccagatctggtacttctttgacaacagcacacagatgaacatgatcagagataccctggggaacccaacagtaaaagactttttgaataggcagcttggtgaagaaggtattactgctgaagccatcctaaacttcctctacaagggccctcgggaaagccaggctgacgacatggccaacttcgactggagggacatatttaacatcactgatcgcaccctccgccttgtcaatcaatacctggagtgcttggtcctggataagtttgaaagctacaatgatgaaactcagctcacccaacgtgccctctctctactggaggaaaacatgttctgggccggagtggtattccctgacatgtatccctggaccagctctctaccaccccacgtgaagtataagatccgaatggacatagacgtggtggagaaaaccaataagattaaagacaggtattgggattctggtcccagagctgatcccgtggaagatttccggtacatctggggcgggtttgcctatctgcaggacatggttgaacaggggatcacaaggagccaggtgcaggcggaggctccagttggaatctacctccagcagatgccctacccctgcttcgtggacgattctttcatgatcatcctgaaccgctgtttccctatcttcatggtgctggcatggatctactctgtctccatgactgtgaagagcatcgtcttggagaaggagttgcgactgaaggagaccttgaaaaatcagggtgtctccaatgcagtgatttggtgtacctggttcctggacagcttctccatcatgtcgatgagcatcttcctcctgacgatattcatcatgcatggaagaatcctacattacagcgacccattcatcctcttcctgttcttgttggctttctccactgccaccatcatgctgtgctttctgctcagcaccttcttctccaaggccagtctggcagcagcctgtagtggtgtcatctatttcaccctctacctgccacacatcctgtgcttcgcctggcaggaccgcatgaccgctgagctgaagaaggctgtgagcttactgtctccggtggcatttggatttggcactgagtacctggttcgctttgaagagcaaggcctggggctgcagtggagcaacatcgggaacagtcccacggaaggggacgaattcagcttcctgctgtccatgcagatgatgctccttgatgctgctgtctatggcttactcgcttggtaccttgatcaggtgtttccaggagactatggaaccccacttccttggtactttcttctacaagagtcgtattggcttggcggtgaagggtgttcaaccagagaagaaagagccctggaaaagaccgagcccctaacagaggaaacggaggatccagagcacccagaaggaatacacgactccttctttgaacgtgagcatccagggtgggttcctggggtatgcgtgaagaatctggtaaagatttttgagccctgtggccggccagctgtggaccgtctgaacatcaccttctacgagaaccagatcaccgcattcctgggccacaatggagctgggaaaaccaccaccttgtccatcctgacgggtctgttgccaccaacctctgggactgtgctcgttgggggaagggacattgaaaccagcctggatgcagtccggcagagccttggcatgtgtccacagcacaacatcctgttccaccacctcacggtggctgagcacatgctgttctatgcccagctgaaaggaaagtcccaggaggaggcccagctggagatggaagccatgttggaggacacaggcctccaccacaagcggaatgaagaggctcaggacctatcaggtggcatgcagagaaagctgtcggttgccattgcctttgtgggagatgccaaggtggtgattctggacgaacccacctctggggtggacccttactcgagacgctcaatctgggatctgctcctgaagtatcgctcaggcagaaccatcatcatgtccactcaccacatggacgaggccgacctccttggggaccgcattgccatcattgcccagggaaggctctactgctcaggcaccccactcttcctgaagaactgctttggcacaggcttgtacttaaccttggtgcgcaagatgaaaaacatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttcc

SEQ ID NO: 10 - DNA, 4203 bp, Artificial Sequence
  example partial downstream vector sequence, comprising CDS, post-transcriptional response element, poly- adenylation sequence
acatccagagccaaaggaaaggcagtgaggggacctgcagctgctcgtctaagggtttctccaccacgtgtccagcccacgtcgatgacctaactccagaacaagtcctggatggggatgtaaatgagctgatggatgtagttctccaccatgttccagaggcaaagctggtggagtgcattggtcaagaacttatcttccttcttccaaataagaacttcaagcacagagcatatgccagccttttcagagagctggaggagacgctggctgaccttggtctcagcagttttggaatttctgacactcccctggaagagatttttctgaaggtcacggaggattctgattcaggacctctgtttgcgggtggcgctcagcagaaaagagaaaacgtcaacccccgacacccctgcttgggtcccagagagaaggctggacagacaccccaggactccaatgtctgctccccaggggcgccggctgctcacccagagggccagcctcccccagagccagagtgcccaggcccgcagctcaacacggggacacagctggtcctccagcatgtgcaggcgctgctggtcaagagattccaacacaccatccgcagccacaaggacttcctggcgcagatcgtgctcccggctacctttgtgtttttggctctgatgctttctattgttatccctccttttggcgaataccccgctttgacccttcacccctggatatatgggcagcagtacaccttcttcagcatggatgaaccaggcagtgagcagttcacggtacttgcagacgtcctcctgaataagccaggctttggcaaccgctgcctgaaggaagggtggcttccggagtacccctgtggcaactcaacaccctggaagactccttctgtgtccccaaacatcacccagctgttccagaagcagaaatggacacaggtcaacccttcaccatcctgcaggtgcagcaccagggagaagctcaccatgctgccagagtgccccgagggtgccgggggcctcccgcccccccagagaacacagcgcagcacggaaattctacaagacctgacggacaggaacatctccgacttcttggtaaaaacgtatcctgctcttataagaagcagcttaaagagcaaattctgggtcaatgaacagaggtatggaggaatttccattggaggaaagctcccagtcgtccccatcacgggggaagcacttgttgggtttttaagcgaccttggccggatcatgaatgtgagcgggggccctatcactagagaggcctctaaagaaatacctgatttccttaaacatctagaaactgaagacaacattaaggtgtggtttaataacaaaggctggcatgccctggtcagctttctcaatgtggcccacaacgccatcttacgggccagcctgcctaaggacaggagccccgaggagtatggaatcaccgtcattagccaacccctgaacctgaccaaggagcagctctcagagattacagtgctgaccacttcagtggatgctgtggttgccatctgcgtgattttctccatgtccttcgtcccagccagctttgtcctttatttgatccaggagcgggtgaacaaatccaagcacctccagtttatcagtggagtgagccccaccacctactgggtaaccaacttcctctgggacatcatgaattattccgtgagtgctgggctggtggtgggcatcttcatcgggtttcagaagaaagcctacacttctccagaaaaccttcctgcccttgtggcactgctcctgctgtatggatgggcggtcattcccatgatgtacccagcatccttcctgtttgatgtccccagcacagcctatgtggctttatcttgtgctaatctgttcatcggcatcaacagcagtgctattaccttcatcttggaattatttgagaataaccggacgctgctcaggttcaacgccgtgctgaggaagctgctcattgtcttcccccacttctgcctgggccggggcctcattgaccttgcactgagccaggctgtgacagatgtctatgcccggtttggtgaggagcactctgcaaatccgttccactgggacctgattgggaagaacctgtttgccatggtggtggaaggggtggtgtacttcctcctgaccctgctggtccagcgccacttcttcctctcccaatggattgccgagcccactaaggagcccattgttgatgaagatgatgatgtggctgaagaaagacaaagaattattactggtggaaataaaactgacatcttaaggctacatgaactaaccaagatttatccaggcacctccagcccagcagtggacaggctgtgtgtcggagttcgccctggagagtgctttggcctcctgggagtgaatggtgccggcaaaacaaccacattcaagatgctcactggggacaccacagtgacctcaggggatgccaccgtagcaggcaagagtattttaaccaatatttctgaagtccatcaaaatatgggctactgtcctcagtttgatgcaatcgatgagctgctcacaggacgagaacatctttacctttatgcccggcttcgaggtgtaccagcagaagaaatcgaaaaggttgcaaactggagtattaagagcctgggcctgactgtctacgccgactgcctggctggcacgtacagtgggggcaacaagcggaaactctccacagccatcgcactcattggctgcccaccgctggtgctgctggatgagcccaccacagggatggacccccaggcacgccgcatgctgtggaacgtcatcgtgagcatcatcagagaagggagggctgtggtcctcacatcccacagcatggaagaatgtgaggcactgtgtacccggctggccatcatggtaaagggcgcctttcgatgtatgggcaccattcagcatctcaagtccaaatttggagatggctatatcgtcacaatgaagatcaaatccccgaaggacgacctgcttcctgacctgaaccctgtggagcagttcttccaggggaacttcccaggcagtgtgcagagggagaggcactacaacatgctccagttccaggtctcctcctcctccctggcgaggatcttccagctcctcctctcccacaaggacagcctgctcatcgaggagtactcagtcacacagaccacactggaccaggtgtttgtaaattttgctaaacagcagactgaaagtcatgacctccctctgcaccctcgagctgctggagccagtcgacaagcccaggactgaaagcttatcgataatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcatgccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctgggg

SEQ ID NO: 11 - DNA, 208 bp, Artificial Sequence
  overlap C sequence with out-of-frame AUG codons prior to an in-frame AUG codon
acauccagagccaaaggaaaggcagugaggggaccugcagcugcucgucuaaggguuucuccaccacguguccagcccacgucgaugaccuaacuccagaacaaguccuggauggggauguaaaugagcugauggauguaguucuccaccauguuccagaggcaaagcugguggagugcauuggucaagaacuuaucuuccuucuucc